(19) United States
(12) Patent Application Publication
(10) Pub. No.: US 2025/0257381 A1
(43) Pub. Date: Aug. 14, 2025

(54) STEVIA REBAUDIANA KAURENOIC ACID HYDROXYLASE VARIANTS FOR HIGH EFFICIENCY PRODUCTION OF REBAUDIOSIDES

(71) Applicant: Amyris, Inc., Emeryville, CA (US)

(72) Inventors: Gale A. WICHMANN, Emeryville, CA (US); Wenzong LI, Emeryville, CA (US); Sean LUND, Emeryville, CA (US); Shaina J. JACKSON, Emeryville, CA (US); Chantal V. GARCIA DE GONZALO, Emeryville, CA (US); Hailley WARBINGTON, Emeryville, CA (US); Yi XIONG, Emeryville, CA (US); Svetlana Alekseyevna BORISOVA, Emeryville, CA (US)

(21) Appl. No.: 19/004,028

(22) Filed: Dec. 27, 2024

Related U.S. Application Data
(62) Division of application No. 17/285,888, filed on Apr. 15, 2021, now Pat. No. 12,221,641.
(60) Provisional application No. 62/745,900, filed on Oct. 15, 2018.

(51) Int. Cl. C12P 19/56; C12N 1/18; C12N 9/02; C12N 9/10; C12N 9/88; C12N 9/90; C12R 1/865

(52) U.S. Cl. CPC C12P 19/56; C12N 1/185; C12N 9/0042; C12N 9/0073; C12N 9/1051; C12N 9/88; C12N 9/90; C07K 2319/03; C12R 2001/865; C12Y 106/02004; C12Y 114/13079; C12Y 114/14; C12Y 204/01017; C12Y 205/01001; C12Y 402/03019; C12Y 505/01012

(57) ABSTRACT

Provided herein are compositions and methods for improved production of steviol glycosides in a host cell. In some embodiments, the host cell is genetically modified to comprise a heterologous nucleotide sequence encoding a Stevia rebaudiana kaurenoic acid hydroxylase. In some embodiments, the host cell further comprises one or more heterologous nucleotide sequence encoding further enzymes of a pathway capable of producing one or more steviol glycosides in the host cell. The compositions and methods described herein provide an efficient route for the heterologous production of steviol glycosides, including but not limited to, rebaudioside D and rebaudioside M.

### CROSS-REFERENCE TO RELATED APPLICATIONS

This application is a Divisional application of U.S. patent application Ser. No. 17/285,888, filed Apr. 15, 2021, which is a National Stage application of PCT Application PCT/US2019/056153, filed Oct. 14, 2019, which claims the benefit of U.S. Provisional Application No. 62/745,900, filed Oct. 15, 2018, the entire contents of each of which are herein incorporated in their entirety for all purposes.

### INCORPORATION BY REFERENCE

This application contains a Sequence Listing which has been submitted electronically in XML format and is hereby incorporated by reference in its entirety. Said. XML copy, created on May 5, 2025, is named “107345.00952.xml” and is 63,887 bytes in size. The sequence listing contained in this .XML file is part of the specification and is hereby incorporated by reference herein in its entirety.

### I. FIELD

The present disclosure relates to certain kaurenoic acid hydroxylases (KAHs), compositions comprising the same, host cells comprising the same, and methods of their use for the production of steviol and/or rebaudiosides including rebaudioside D and rebaudioside M.

### II. BACKGROUND

Zero-calorie sweeteners derived from natural sources are desired to limit the ill effects of high-sugar consumption (e.g., diabetes and obesity). Rebaudioside M (RebM), is one of many sweet-tasting compounds produced by the stevia plant (S. rebaudiana bertoni). Of all the rebaudiosides, RebM has the highest potency (˜200-300× sweeter than sucrose) and is the cleanest tasting. However, RebM is only produced in minor quantities by the Stevia plant and is a small fraction of the total steviol glycoside content (<1.0%). Ohta et al., 2010, J. Appl. Glycosci., 57, 199-209 (2010). As such, it is desirable to produce RebM using biotechnological routes allowing production in large quantities and at high purity.

To economically produce a product using biotechnology, each step in the bioconversion from feedstock to product needs to have a high conversion efficiency (ideally >90%). In our engineering of yeast to produce RebM, we identified a limitation in the biosynthetic step early in the pathway to RebM that takes kaurenoic acid to steviol (FIG. 1).

The kaurenoic acid hydroxylase (KAH) enzyme is found in the plant Stevia rebaudiana and normally acts to produce the C20 isoprenoid steviol from the plant hormone-precursor kaurenoic acid. Even though S. rebaudiana can accumulate up to approximately 15% of leaf dry weight in steviol glycosides, the flux through the KAH enzyme may not be what is required for high volume RebM production in yeast for commercial manufacturing. Conventionally, the wild type KAH enzyme from Stevia rebaudiana (Sr.KAH) has been used to convert kaurenoic acid to steviol in yeast engineered to produce RebM.

To produce RebM efficiently and at high purity, improved enzymes capable of producing steviol at high efficiency are needed. The compositions and methods provided herein address this need and provide related advantages as well.

### III. SUMMARY

Provided herein are compositions and methods for the improved conversion of kaurenoic acid to steviol. These compositions and methods are based in part on the production of certain kaurenoic acid hydroxylases (KAHs) that are capable of converting kaurenoic acid to steviol with remarkably high efficiency. Even a modest improvement in strain performance (e.g., ten percent) with new KAHs can potentially save over ten million dollars in production cost in the future, assuming that the market demand for RebM is 5000 million tons per year.

Certain KAHs described herein are also capable of producing steviol with little or no residual kaurenoic acid. As such, in certain embodiments, the compositions and methods described herein can reduce the costs of downstream processing to obtain a composition with high yield steviol glycosides such as RebM.

In one aspect, provided herein are genetically modified host cells and methods of their use for the production of industrially useful compounds. In one aspect, provided herein is a genetically modified host cell comprising: a heterologous nucleic acid encoding a Stevia rebaudiana kaurenoic acid hydroxylase provided herein. In some embodiments, the genetically modified host cell further comprises one or more enzymatic pathways capable of producing steviol and/or steviol glycosides. In certain embodiments, the genetically modified host cell is capable of converting kaurenoic acid to steviol at an efficiency greater than 50%, 55%, 60%, 65%, 70%, 75%, 80%, 85%, 90%, 95%, 96%, 97%, or 98%.

In another aspect, provided herein are methods for producing a heterologous steviol glycoside, the method comprising: culturing a population of genetically modified host cells provided herein, capable of producing the heterologous steviol glycoside as described herein, in a medium with a carbon source under conditions suitable for making said heterologous steviol glycoside compound; and recovering said steviol glycoside from the medium. In some embodiments, heterologous steviol glycoside is selected from the group consisting of RebD and RebM.

In another aspect, provided herein are methods for producing RebD, the method comprising: culturing a population of genetically modified host cells provided herein, capable of producing RebD as described herein, in a medium with a carbon source under conditions suitable for making said RebD; and recovering said RebD from the medium.

In another aspect, provided herein are methods for producing RebM, the method comprising: culturing a population of genetically modified host cells provided herein, capable of producing RebM as described herein, in a medium with a carbon source under conditions suitable for making said RebM; and recovering said RebM from the medium.

In another aspect, provided herein are methods for producing steviol, the method comprising: contacting kaurenoic acid with a kaurenoic acid hydroxylase described herein, capable of converting kaurenoic acid to steviol, under conditions suitable for forming steviol.

In some embodiments, the host cell is a yeast cell. In some embodiments, the yeast is Saccharomyces cerevisiae. In some embodiments, the host cell produces RebD or RebM at high efficiency. In some embodiments, the host cell produces an increased amount of RebD or RebM compared to a yeast cell not comprising the Stevia rebaudiana kaurenoic acid hydroxy lase polypeptide provided herein.

### IV. BRIEF DESCRIPTION OF THE FIGURES

FIG. 1 provides an enzymatic pathway from the native yeast metabolite farnesyl pyrophosphate (FPP) to Rebaudioside M (RebM). Depicted in the figure are geranylgeranyl pyrophosphate (GGPP), copalyl pyrophosphate (CPP), and several rebaudiosides (Reb).

FIG. 2 provides a schematic diagram of a “landing pad” design used to insert individual KAH enzymes for screening for kaurenoic acid to steviol conversion in yeast.

FIG. 3 provides Sr.KAH mutants, each containing a single amino acid change, that have activity at least one standard deviation higher than the wild type Sr.KAH allele. The Y-axis represents the ratio of 19-glycoside (Strain 1) or Reb M (Strain 2) produced by Sr.KAH variant to that of wild type Sr.KAH.

FIG. 4 provides combinations of Sr.KAH protein mutations leading to improvement of in vivo activity from 4.3× to 6.3× in a yeast strain; Sr.KAH activity is measured in the Strain 3 background. The Y-axis is the relative fold increase of mutant Sr.KAH alleles over wild type Sr.KAH; wild type Sr.KAH activity is normalized to one.

FIG. 5 provides improved KAH mutants leading to a reduction in the substrate kaurenoic acid compared to wild type Sr.KAH demonstrating that Sr.KAH alleles with improved activity are converting more substrate to steviol.

FIG. 6 provides in vivo KAH activity of the degenerate codon library mutants measured in a Tier 1 screen versus the Sr.KAH mutant #3 allele using the titer of total steviol glycosides (μM) as described in Example 7.

### V. DETAILED DESCRIPTION OF THE EMBODIMENTS

### 5.1 Terminology

As used herein, the term “heterologous” refers to what is not normally found in nature. The term “heterologous nucleotide sequence” refers to a nucleotide sequence not normally found in a given cell in nature. As such, a heterologous nucleotide sequence may be: (a) foreign to its host cell (i.e., is “exogenous” to the cell); (b) naturally found in the host cell (i.e., “endogenous”) but present at an unnatural quantity in the cell (i.e., greater or lesser quantity than naturally found in the host cell); or (c) be naturally found in the host cell but positioned outside of its natural locus.

On the other hand, the term “native” or “endogenous” as used herein with reference to molecules, and in particular enzymes and nucleic acids, indicates molecules that are expressed in the organism in which they originated or are found in nature. It is understood that expression of native enzymes or polynucleotides may be modified in recombinant microorganisms.

As used herein, the term “parent cell” refers to a cell that has an identical genetic background as a genetically modified host cell disclosed herein except that it does not comprise one or more particular genetic modifications engineered into the modified host cell, for example, one or more modifications selected from the group consisting of: heterologous expression of an enzyme of a steviol pathway, heterologous expression of an enzyme of a steviol glycoside pathway, heterologous expression of a geranylgeranyl diphosphate synthase, heterologous expression of a copalyl diphosphate synthase, heterologous expression of a kaurene synthase, heterologous expression of a kaurene oxidase (e.g., Pisum sativum kaurene oxidase), heterologous expression of a steviol synthase (kaurenoic acid hydroxylase), heterologous expression of a cytochrome P450 reductase, heterologous expression of a UGT74G1, heterologous expression of a UGT76G1, heterologous expression of a UGT85C2, heterologous expression of a UGT91D, and heterologous expression of a UGT40087 or its variant.

As used herein, the term “naturally occurring” refers to what is found in nature. For example, a kaurenoic acid hydroxylase that is present in an organism that can be isolated from a source in nature and that has not been intentionally modified by a human in the laboratory is naturally occurring kaurenoic acid hydroxylase. Conversely, as used herein, the term “non-naturally occurring” refers to what is not found in nature but is created by human intervention.

The term “medium” refers to a culture medium and/or fermentation medium.

The term “fermentation composition” refers to a composition which comprises genetically modified host cells and products or metabolites produced by the genetically modified host cells. An example of a fermentation composition is a whole cell broth, which can be the entire contents of a vessel (e.g., a flask, plate, or fermentor), including cells, aqueous phase, and compounds produced from the genetically modified host cells.

As used herein, the term “production” generally refers to an amount of steviol or steviol glycoside produced by a genetically modified host cell provided herein. In some embodiments, production is expressed as a yield of steviol or steviol glycoside by the host cell. In other embodiments, production is expressed as the productivity of the host cell in producing the steviol or steviol glycoside.

As used herein, the term “productivity” refers to production of a steviol or steviol glycoside by a host cell, expressed as the amount of steviol or steviol glycoside produced (by weight) per amount of fermentation broth in which the host cell is cultured (by volume) over time (per hour).

As used herein, the term “yield” refers to production of a steviol or steviol glycoside by a host cell, expressed as the amount of steviol or steviol glycoside produced per amount of carbon source consumed by the host cell, by weight.

As used herein, the term “an undetectable level” of a compound (e.g., RebM, steviol glycosides, or other compounds) means a level of a compound that is too low to be measured and/or analyzed by a standard technique for measuring the compound. For instance, the term includes the level of a compound that is not detectable by the analytical methods known in the art.

The term “kaurene” refers to the compound kaurene, including any stereoisomer of kaurene. In particular embodiments, the term refers to the enantiomer known in the art as ent-kaurene. In particular embodiments, the term refers to the compound according to the following structure:

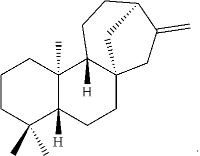

The term “kaurenol” refers to the compound kaurenol, including any stereoisomer of kaurenol. In particular embodiments, the term refers to the enantiomer known in the art as ent-kaurenol. In particular embodiments, the term refers to the compound according to the following structure.

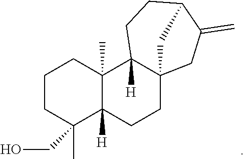

The term “kaurenal” refers to the compound kaurenal, including any stereoisomer of kaurenal. In particular embodiments, the term refers to the enantiomer known in the art as ent-kaurenal. In particular embodiments, the term refers to the compound according to the following structure.

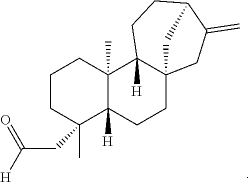

The term “kaurenoic acid” refers to the compound kaurenoic acid, including any stereoisomer of kaurenoic acid. In particular embodiments, the term refers to the enantiomer known in the art as ent-kaurenoic acid. In particular embodiments, the term refers to the compound according to the following structure.

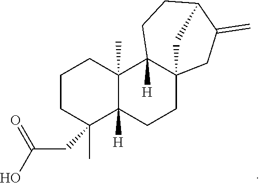

The term “steviol” refers to the compound steviol, including any stereoisomer of steviol. In particular embodiments, the term refers to the compound according to the following structure.

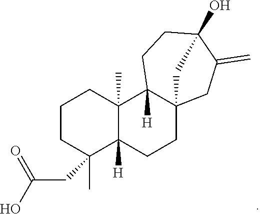

As used herein, the term “steviol glycoside(s)” refers to a glycoside of steviol, including, but not limited to, naturally occurring steviol glycosides, e.g. steviolmonoside, steviolbioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, synthetic steviol glycosides, e.g. enzymatically glycosylated steviol glycosides and combinations thereof.

As used herein, the term “variant” refers to a polypeptide differing from a specifically recited “reference” polypeptide (e.g., a wild-type sequence) by amino acid insertions, deletions, mutations, and/or substitutions, but retains an activity that is substantially similar to the reference polypeptide. In some embodiments, the variant is created by recombinant DNA techniques or by mutagenesis. In some embodiments, a variant polypeptide differs from its reference polypeptide by the substitution of one basic residue for another (i.e. Arg for Lys), the substitution of one hydrophobic residue for another (i.e. Leu for Ile), or the substitution of one aromatic residue for another (i.e. Phe for Tyr), etc. In some embodiments, variants include analogs wherein conservative substitutions resulting in a substantial structural analogy of the reference sequence are obtained. Examples of such conservative substitutions, without limitation, include glutamic acid for aspartic acid and vice-versa; glutamine for asparagine and vice-versa; serine for threonine and vice-versa; lysine for arginine and vice-versa; or any of isoleucine, valine or leucine for each other.

As used herein, the term “sequence identity” or “percent identity,” in the context or two or more nucleic acid or protein sequences, refers to two or more sequences or subsequences that are the same or have a specified percentage of amino acid residues or nucleotides that are the same. For example, the sequence can have a percent identity of at least 60%, at least 65%, at least 70%, at least 75%, at least 80%, at least 85%, at least 90%, at least 91% at least 92%, at least 93%, at least 94%, at least 95%, at least 96%, at least 97%, at least 98%, at least 99%, or higher identity over a specified region to a reference sequence when compared and aligned for maximum correspondence over a comparison window, or designated region as measured using a sequence comparison algorithm or by manual alignment and visual inspection. For example, percent of identity is determined by calculating the ratio of the number of identical nucleotides (or amino acid residues) in the sequence divided by the length of the total nucleotides (or amino acid residues) minus the lengths of any gaps.

For convenience, the extent of identity between two sequences can be ascertained using computer programs and mathematical algorithms known in the art. Such algorithms that calculate percent sequence identity generally account for sequence gaps and mismatches over the comparison region. Programs that compare and align sequences, like Clustal W (Thompson et al., (1994) Nucleic Acids Res., 22: 4673-4680), ALIGN (Myers et al., (1988) CABIOS, 4: 11-17), FASTA (Pearson et al., (1988) PNAS, 85:2444-2448; Pearson (1990), Methods Enzymol., 183: 63-98) and gapped BLAST (Altschul et al., (1997) Nucleic Acids Res., 25: 3389-3402) are useful for this purpose. The BLAST or BLAST 2.0 (Altschul et al., J. Mol. Biol. 215:403-10, 1990) is available from several sources, including the National Center for Biological Information (NCBI) and on the Internet, for use in connection with the sequence analysis programs BLASTP, BLASTN, BLASTX, TBLASTN, and TBLASTX. Additional information can be found at the NCBI web site.

In certain embodiments, the sequence alignments and percent identity calculations can be determined using the BLAST program using its standard, default parameters. For nucleotide sequence alignment and sequence identity calculations, the BLASTN program is used with its default parameters (Gap opening penalty=5, Gap extension penalty=2, Nucleic match=2, Nucleic mismatch=−3, Expectation value=10.0, Word size=11, Max matches in a query range=0). For polypeptide sequence alignment and sequence identity calculations, BLASTP program is used with its default parameters (Alignment matrix=BLOSUM62; Gap costs: Existence=11, Extension=1; Compositional adjustments=Conditional compositional score, matrix adjustment; Expectation value=10.0; Word size=6; Max matches in a query range=0). Alternatively, the following program and parameters can be used: Align Plus software of Clone Manager Suite, version 5 (Sci-Ed Software); DNA comparison: Global comparison, Standard Linear Scoring matrix, Mismatch penalty=2. Open gap penalty=4, Extend gap penalty=1. Amino acid comparison: Global comparison, BLOSUM 62 Scoring matrix. In the embodiments described herein, the sequence identity is calculated using BLASTN or BLASTP programs using their default parameters. In the embodiments described herein, the sequence alignment of two or more sequences are performed using Clustal W using the suggested default parameters (Dealign input sequences: no; Mbed-like clustering guide-tree: yes; Mbed-like clustering iteration: yes; number of combined iterations: default(0); Max guide tree iterations: default; Max HMM iterations: default; Order: input).

### 5.2 Kaurenoic Acid Hydroxylase Polypeptides, Nucleic Acids, and Host Cells

In one aspect, provided herein are modified kaurenoic acid hydroxylase polypeptides which include modification(s) of one or more amino acid residues compared to a wild-type kaurenoic acid hydroxylase polypeptide. In certain embodiments the wild-type kaurenoic acid hydroxylase is Stevia rebaudiana kaurenoic acid hydroxylase, i.e. Sr.KAH. In certain embodiments, the wild-type kaurenoic acid hydroxylase polypeptide has the amino acid sequence provided in SEQ ID NO: 1. In certain embodiments, the actual residue numbers can be determined by standard alignment techniques relative to SEQ ID NO: 1, including those described herein. In particular embodiments, provided herein are the nucleic acids encoding the polypeptides. In particular embodiments, the polypeptides retain activity as a kaurenoic acid hydroxylase to convert kaurenoic acid to steviol. In preferred embodiments, the modified kaurenoic acid hydroxylase polypeptides have improved activity, for instance, compared to Sr.KAH.

Also provided herein are host cells comprising one or more of the kaurenoic acid hydroxy lase polypeptides or nucleic acids provided herein. In certain embodiments, the host cells can produce steviol from kaurenoic acid as a starting material. In particular embodiments, the host cells can produce steviol from a carbon source in a culture medium. In particular embodiments, the host cells can produce steviol from a carbon source in a culture medium and can further produce RebA or RebD from the steviol. In particular embodiments, the host cells can further produce rebaudioside M (RebM) from the RebD.

In certain embodiments, provided herein is a kaurenoic acid hydroxylase polypeptide comprising one, two, three, four, five, six, seven, eight, nine, ten, or eleven of the following mutations: I166R, I153L, S158D, G306L, L232D, I333V, I350L, V316L, G447V, M308L. In certain embodiments, the residue numbers of the polypeptide are according to SEQ ID NO:1. In certain embodiments, provided herein is a polypeptide homologous to SEQ ID NO: 1 and comprising one, two, three, four, five, six, seven, eight, nine, ten, or eleven of the following mutations: I166R, I153L, S158D, G306L, L232D, I333V, I350L, V316L, G447V, M308L. In any of the previous embodiments, the polypeptide comprises a heterologous amino terminal domain.

In certain embodiments, provided herein a polypeptide homologous to SEQ ID NO: 1, residues numbers are provided with respect to SEQ ID NO: 1 where actual residue numbers can be determined by standard alignment techniques relative to SEQ ID NO:1, including those described herein.

In certain embodiments, provided herein is a kaurenoic acid hydroxylase polypeptide comprising the following mutations: I166R and I333V. In certain embodiments, the residue numbers are according to SEQ ID NO: 1.

In certain embodiments, provided herein is a kaurenoic acid hydroxy lase polypeptide comprising the following mutations: I166R, I350L, and G447V. In certain embodiments, the residue numbers are according to SEQ ID NO: 1.

In certain embodiments, provided herein is a kaurenoic acid hydroxylase polypeptide comprising the following mutations: I153L, S158D, I166R, L232D, I333V, and I350L. In certain embodiments, the residue numbers are according to SEQ ID NO: 1.

In any of the previous embodiments, provided herein the kaurenoic acid hydroxy lase polypeptide further comprises one or more mutations selected from E492C, M427A, G306D, V333C, D191L, I40S, L445I, S114A, D191Y, D191F, L497R, G306L, N29G, T167G, T164S, Q415H, T89A, L13D, R258T, and L13V. In certain embodiments, the residue numbers are according to SEQ ID NO: 1.

In any of the previous embodiments, provided herein the kaurenoic acid hydroxy lase polypeptide further comprises one or more mutations selected from L13D, R258F, and L13V. In certain embodiments, the residue numbers are according to SEQ ID NO: 1.

In certain embodiments, provided herein is a kaurenoic acid hydroxylase polypeptide comprising the following mutations: S158D, G306L, and I350L. In certain embodiments, the residue numbers are according to SEQ ID NO: 1.

In certain embodiments, provided herein is a kaurenoic acid hydroxylase polypeptide comprising the following mutations: G306L, V316L, I350L, and G447V. In certain embodiments, the residue numbers are according to SEQ ID NO:1.

In certain embodiments, provided herein is a kaurenoic acid hydroxylase polypeptide comprising the following mutations: G306L, V316L, I333V, and I350L. In certain embodiments, the residue numbers are according to SEQ ID NO:1.

In certain embodiments, provided herein is a kaurenoic acid hydroxylase polypeptide comprising the following mutations: S158D, I166R, L232D, G306L, and I333V. In certain embodiments, the residue numbers are according to SEQ ID NO: 1.

In certain embodiments, provided herein is a kaurenoic acid hydroxylase polypeptide comprising the following mutations: L232D, G306L, V316L, I333V, and I350L. In certain embodiments, the residue numbers are according to SEQ ID NO: 1.

In certain embodiments, provided herein is a kaurenoic acid hydroxylase polypeptide comprising the following mutations: I166R, L232D, G306L, and I350L. In certain embodiments, the residue numbers are according to SEQ ID NO: 1.

In certain embodiments, provided herein is a kaurenoic acid hydroxylase polypeptide comprising the following mutations: S158D, I166R, G306L, M308L, V316L, and I350L. In certain embodiments, the residue numbers are according to SEQ ID NO: 1.

In certain embodiments, provided herein is a kaurenoic acid hydroxy lase polypeptide comprising at least one of the following mutations: Y62H, T164R, L76V, Q415L, A60Y, S182C, T167N, Y52H, R266D, T167H, I153L, K100L, G306C, Q120N, L232H, I333V, G351R, L232S, I166S, W447C, I443Y, I166N, N355Y, L232D, S158D, A442G, G306N, V316L, M308L, G447F, G306I, G306V, I350L, G447V, I166R, and G306L. In certain embodiments, the residue numbers are according to SEQ ID NO: 1.

In certain embodiments, provided herein is a kaurenoic acid hydroxylase polypeptide of any of the previous embodiments, further comprising at least one of the following mutations: E492C, M427A, G306D, V333C, D191L, I40S, L445I, S114A, D191Y, D191F, L497R, G306L, N29G, T167G, T164S, Q415H, T89A, L13D. R258T, and L13V. In certain embodiments, the residue numbers are according to SEQ ID NO: 1.

In certain embodiments, provided herein is a chimeric kaurenoic acid hydroxylase polypeptide comprising a catalytic domain of a Stevia rebaudiana kaurenoic acid hydroxylase, and an N-terminal transmembrane domain of an endoplasmic reticulum (ER) bound protein, wherein the N-terminal transmembrane domain is covalently linked to the catalytic domain. In still further embodiments, the chimeric kaurenoic acid hydrolase polypeptide further comprises at least one of the following mutations: Y62H, T164R, L76V, Q415L, A60Y, S182C, T167N, Y52H, R266D, T167H, I153L, K100L, G306C, Q120N, L232H, I333V, G351R, L232S, I166S, W447C, I443Y, I166N, N355Y, L232D, S158D, A442G, G306N, V316L, M308L, G447F, G306I, G306V, I350L, G447V, I166R, and G306L. In certain embodiments, the residue numbers are according to SEQ ID NO:1.

In certain embodiments, provided herein is a chimeric kaurenoic acid hydroxylase polypeptide comprising a catalytic domain of a Stevia rebaudiana kaurenoic acid hydroxylase, and an N-terminal transmembrane domain of ATR2, wherein the N-terminal transmembrane domain is covalently linked to the catalytic domain.

In certain embodiments, provided herein is a chimeric kaurenoic acid hydroxylase polypeptide comprising a catalytic domain of a Stevia rebaudiana kaurenoic acid hydroxylase wherein the catalytic domain comprises amino acids 23 through 500 of the Stevia rebaudiana kaurenoic acid hydroxylase, and an N-terminal transmembrane domain of ATR2, wherein the N-terminal domain comprises amino acids 1 through 72, and wherein the N-terminal transmembrane domain is covalently linked to the catalytic domain. In certain embodiments, the N-terminal domain of the chimeric kaurenoic acid hydroxylase comprises amino acids 1 through 72 of SEQ ID No. 22. In certain embodiments, the catalytic domain comprises amino acids 23 through 500 of SEQ ID No. 1.

In certain embodiments, provided herein the catalytic domain comprises amino acids 5 through 500 of the Stevia rebaudiana kaurenoic acid hydroxylase.

In certain embodiments, provided herein the catalytic domain comprises amino acids 23 through 500 of the Stevia rebaudiana kaurenoic acid hydroxylase.

In certain embodiments, provided herein the catalytic domain comprises amino acids 47 through 500 of the Stevia rebaudiana kaurenoic acid hydroxylase.

In certain embodiments, the N-terminal domain of the chimeric kaurenoic acid hydroxy lase polypeptide comprises amino acids 1 through 72 of the Arabidopsis thaliana ATR2 protein. In certain embodiments, the N-terminal domain comprises amino acids 1 through 72 of SEQ ID NO. 22.

In certain embodiments, the N-terminal domain of the chimeric kaurenoic acid hydroxy lase polypeptide comprises amino acids 1 through 50 of the Arabidopsis thaliana ATR2 protein. In certain embodiments, the N-terminal domain comprises amino acids 1 through 50 of SEQ ID NO. 22.

In any of the previous embodiment, the chimeric kaurenoic acid hydroxylase polypeptide further comprises at least one mutation at kaurenoic acid hydroxylase positions E492C, M427A, G306D, V333C, D191L, I40S, L445I, S114A, D191Y, D191F, L497R, G306L, N29G, T167G, T164S, Q415H, T89A, L13D, R258T, and L13V, according to SEQ ID NO. 1.

In certain embodiments, provided herein is a kaurenoic acid hydroxylase polypeptide comprising a heterologous amino terminal domain. In certain embodiments, the kaurenoic acid hydroxylase polypeptide comprising a heterologous amino terminal domain is also known as a chimeric kaurenoic acid hydroxy lase polypeptide.

In certain embodiments, provided herein is a substituted amino terminal segment of a kaurenoic acid hydroxylase polypeptide that is substituted with a heterologous amino terminal domain. In some embodiments, provided herein the substituted amino terminal segment (the segment removed) comprises 4, 22, or 46 amino acids from the N-terminus of a full length kaurenoic acid hydroxylase polypeptide according to the residue positions of SEQ ID NO: 1. In some embodiments, provided herein the substituted amino terminal segment comprises a deletion of amino acids 1 through 4 from the N-terminus of the kaurenoic acid hydroxylase polypeptide, according to the residue positions of SEQ ID NO: 1. In some embodiments, provided herein the substituted amino terminal segment comprises a deletion of amino acids 1 through 5 from the N-terminus of the kaurenoic acid hydroxylase polypeptide, according to the residue positions of SEQ ID NO: 1. In some embodiments, provided herein the substituted amino terminal segment comprises a deletion of amino acids 1 through 10 from the N-terminus of the kaurenoic acid hydroxylase polypeptide, according to the residue positions of SEQ ID NO: 1. In some embodiments, provided herein the substituted amino terminal segment comprises a deletion of amino acids 1 through 15 from the N-terminus of the kaurenoic acid hydroxylase polypeptide, according to the residue positions of SEQ ID NO: 1. In some embodiments, provided herein the substituted amino terminal segment comprises a deletion of amino acids 1 through 20 from the N-terminus of the kaurenoic acid hydroxylase polypeptide, according to the residue positions of SEQ ID NO: 1. In some embodiments, provided herein the substituted amino terminal segment comprises a deletion of amino acids 1 through 22 from the N-terminus of the kaurenoic acid hydroxylase polypeptide, according to the residue positions of SEQ ID NO: 1. In some embodiments, provided herein the substituted amino terminal segment comprises a deletion of amino acids 1 through 25 from the N-terminus of the kaurenoic acid hydroxylase polypeptide, according to the residue positions of SEQ ID NO: 1. In some embodiments, provided herein the substituted amino terminal segment comprises a deletion of amino acids 1 through 30 from the N-terminus of the kaurenoic acid hydroxylase polypeptide, according to the residue positions of SEQ ID NO: 1. In some embodiments, provided herein the substituted amino terminal segment comprises a deletion of amino acids 1 through 35 from the N-terminus of the kaurenoic acid hydroxylase polypeptide, according to the residue positions of SEQ ID NO: 1. In some embodiments, provided herein the substituted amino terminal segment comprises a deletion of amino acids 1 through 40 from the N-terminus of the kaurenoic acid hydroxylase polypeptide, according to the residue positions of SEQ ID NO: 1. In some embodiments, provided herein the substituted amino terminal segment comprises a deletion of amino acids 1 through 45 from the N-terminus of the kaurenoic acid hydroxylase polypeptide, according to the residue positions of SEQ ID NO: 1. In some embodiments, provided herein the substituted amino terminal segment comprises a deletion of amino acids 1 through 46 from the N-terminus of the kaurenoic acid hydroxylase polypeptide, according to the residue positions of SEQ ID NO: 1.

In some embodiments, provided herein the substituted heterologous amino terminal domain (the segment added) is from a heterologous protein. In some embodiments, provided herein the substituted heterologous amino terminal domain is an N-terminal transmembrane domain of an endoplasmic reticulum (ER) bound protein. In some embodiments, provided herein the substituted heterologous amino terminal domain is from ATR2 (Arabidopsis thaliana cytochrome P450 reductase), Aa.CPR (Artemisia annua), Sr.KO (Stevia rebaudiana kaurene oxidase), ERG11 (Saccharomyces cerevisiae membrane protein), ALG11 (Saccharomyces cerevisiae membrane protein), SEC66 (Saccharomyces cerevisiae membrane protein), NUS1 (Saccharomyces cerevisiae membrane protein), RCR1 (Saccharomyces cerevisiae membrane protein), or UBP1 (Saccharomyces cerevisiae membrane protein).

In certain embodiments, the heterologous amino terminal segment is selected from the amino terminal of any of SEQ ID NOs: 33-40. In some embodiments, provided herein the substituted heterologous amino terminal domain comprises amino acids 1 through 10 of the heterologous protein. In some embodiments, provided herein the substituted heterologous amino terminal domain comprises amino acids 1 through 20 of the heterologous protein. In some embodiments, provided herein the substituted heterologous amino terminal domain comprises amino acids 1 through 23 of the heterologous protein. In some embodiments, provided herein the substituted heterologous amino terminal domain comprises amino acids 1 through 25 of the heterologous protein. In some embodiments, provided herein the substituted heterologous amino terminal domain comprises amino acids 1 through 30 of the heterologous protein. In some embodiments, provided herein the substituted heterologous amino terminal domain comprises amino acids 1 through 35 of the heterologous protein. In some embodiments, provided herein the substituted heterologous amino terminal domain comprises amino acids 1 through 40 of the heterologous protein. In some embodiments, provided herein the substituted heterologous amino terminal domain comprises amino acids 1 through 45 of the heterologous protein. In some embodiments, provided herein the substituted heterologous amino terminal domain comprises amino acids 1 through 50 of the heterologous protein. In some embodiments, provided herein the substituted heterologous amino terminal domain comprises amino acids 1 through 51 of the heterologous protein. In some embodiments, provided herein the substituted heterologous amino terminal domain comprises amino acids 1 through 52 of the heterologous protein. In some embodiments, provided herein the substituted heterologous amino terminal domain comprises amino acids 1 through 55 of the heterologous protein. In some embodiments, provided herein the substituted heterologous amino terminal domain comprises amino acids 1 through 60 of the heterologous protein. In some embodiments, provided herein the substituted heterologous amino terminal domain comprises amino acids 1 through 62 of the heterologous protein. In some embodiments, provided herein the substituted heterologous amino terminal domain comprises amino acids 1 through 65 of the heterologous protein. In some embodiments, provided herein the substituted heterologous amino terminal domain comprises amino acids 1 through 66 of the heterologous protein. In some embodiments, provided herein the substituted heterologous amino terminal domain comprises amino acids 1 through 70 of the heterologous protein. In some embodiments, provided herein the substituted heterologous amino terminal domain comprises amino acids 1 through 72 of the heterologous protein. In some embodiments, provided herein the substituted heterologous amino terminal domain comprises amino acids 1 through 75 of the heterologous protein. In some embodiments, provided herein the substituted heterologous amino terminal domain comprises amino acids 1 through 80 of the heterologous protein. In some embodiments, provided herein the substituted heterologous amino terminal domain comprises amino acids 1 through 85 of the heterologous protein. In some embodiments, provided herein the substituted heterologous amino terminal domain comprises amino acids 1 through 90 of the heterologous protein. In some embodiments, provided herein the substituted heterologous amino terminal domain comprises amino acids 1 through 95 of the heterologous protein. In some embodiments, provided herein the substituted heterologous amino terminal domain comprises amino acids 1 through 100 of the heterologous protein. In some embodiments, provided herein the substituted heterologous amino terminal domain comprises amino acids 1 through 105 of the heterologous protein. In some embodiments, provided herein the substituted heterologous amino terminal domain comprises amino acids 1 through 110 of the heterologous protein. In some embodiments, provided herein the substituted heterologous amino terminal domain comprises amino acids 1 through 115 of the heterologous protein. In some embodiments, provided herein the substituted heterologous amino terminal domain comprises amino acids 1 through 119 of the heterologous protein.

In certain embodiments, provided herein is a kaurenoic acid hydroxylase polypeptide comprising a heterologous amino terminal segment. In certain embodiments, an amino terminal segment of a kaurenoic acid hydroxylase polypeptide is substituted with a heterologous amino terminal domain. In some embodiments, the substituted amino terminal segment (the segment removed) has from 4-42 amino acids. In some embodiments, the substituted amino terminal segment has 4, 22, 38, or 42 amino acids. In some embodiments, the heterologous amino terminal segment (the segment added, replacing the removed segment) is from ATR2 (Arabidopsis thaliana cytochrome P450 reductase), CYP816 (a cytochrome P450 from Santalum album), or Sr.KO (Stevia rebaudiana kaurene oxidase). In certain embodiments, the heterologous amino terminal segment is selected from the amino terminal segment of any of SEQ ID NOs: 2-6. In certain embodiments. 22 amino terminal amino acids are substituted with the amino terminal segment of SEQ ID NO:2. In certain embodiments, four amino terminal amino acids are substituted with the amino terminal segment of SEQ ID NO:3. In certain embodiments, 42 amino terminal amino acids are substituted with the amino terminal segment of SEQ ID NO:4. In certain embodiments, four amino terminal amino acids are substituted with the amino terminal segment of SEQ ID NO: 5. In certain embodiments, 38 amino terminal amino acids are substituted with the amino terminal segment of SEQ ID NO:6. In certain embodiments, any substitution of this paragraph is combined with any one of more of the mutations described above.

In certain embodiments, the kaurenoic acid hydroxylase polypeptide according to the above embodiments produces at least 1.5-fold, at least 2.0-fold, at least 2.5-fold, at least 3.0-fold, at least 3.5-fold, at least 4.0-fold, at least 4.5-fold, at least 5.0-fold, at least 5.5-fold, or at least 6.0-fold more steviol and steviol glycosides compared to wild-type Stevia rebaudiana kaurenoic acid hydroxylase polypeptide according to SEQ ID NO:1.

In certain embodiments, the modified Stevia rebaudiana kaurenoic acid hydroxylase polypeptide is capable of converting kaurenoic acid to steviol at high efficiency. In certain embodiments, the modified Stevia rebaudiana kaurenoic acid hydroxylase polypeptide is capable of converting kaurenoic acid to steviol at an efficiency of greater than 30%. In certain embodiments, the modified Stevia rebaudiana kaurenoic acid hydroxylase polypeptide is capable of converting kaurenoic acid to steviol at an efficiency of greater than 35%. In certain embodiments, the modified Stevia rebaudiana kaurenoic acid hydroxylase polypeptide is capable of converting kaurenoic acid to steviol at an efficiency of greater than 40%. In certain embodiments, the modified Stevia rebaudiana kaurenoic acid hydroxylase polypeptide is capable of converting kaurenoic acid to steviol at an efficiency of greater than 50%. In certain embodiments, the modified Stevia rebaudiana kaurenoic acid hydroxylase polypeptide is capable of converting kaurenoic acid to steviol at an efficiency of greater than 60%. In certain embodiments, the modified Stevia rebaudiana kaurenoic acid hydroxylase polypeptide is capable of converting kaurenoic acid to steviol at an efficiency of greater than 70%. In certain embodiments, the modified Stevia rebaudiana kaurenoic acid hydroxylase polypeptide is capable of converting kaurenoic acid to steviol at an efficiency of greater than 80%. In certain embodiments, the modified Stevia rebaudiana kaurenoic acid hydroxylase polypeptide is capable of converting kaurenoic acid to steviol at an efficiency of greater than 90%. In certain embodiments, the modified Stevia rebaudiana kaurenoic acid hydroxylase polypeptide is capable of converting kaurenoic acid to steviol at an efficiency of greater than 95%. In certain embodiments, the modified Stevia rebaudiana kaurenoic acid hydroxylase polypeptide is capable of converting kaurenoic acid to steviol at an efficiency of greater than 60%, 65%, 70%, 75%, 80%, 85%, 90%, or 95%.

In certain embodiments, the host cell is capable of converting kaurenoic acid to steviol at an efficiency of greater than 30%. In certain embodiments, the host cell is capable of converting kaurenoic acid to steviol an efficiency of greater than 35%. In certain embodiments, the host cell is capable of converting kaurenoic acid to steviol at an efficiency of greater than 40%. In certain embodiments, the host cell is capable of converting kaurenoic acid to steviol at an efficiency of greater than 45%. In certain embodiments, the host cell is capable of converting kaurenoic acid to steviol at an efficiency of greater than 50%. In certain embodiments, the host cell is capable of converting kaurenoic acid to steviol at an efficiency of greater than 55%. In certain embodiments, the host cell is capable of converting kaurenoic acid to steviol at an efficiency of about 58%. In certain embodiments, the host cell is capable of converting kaurenoic acid to steviol at an efficiency of greater than 60%, 65%, 70%, 75%, 80%, 85%, 90%, or 95%.

Efficiency of conversion can be measured by any technique apparent to those of skill in the art. In certain embodiments, efficiency of conversion can be measured by contacting kaurenoic acid with an enzyme or host cell under suitable conditions for forming steviol. Efficiency can be measured by comparing the molar amount of steviol produced compared to the total amount of kaurenoic acid in the resulting composition. Efficiency can also be measured by comparing the total amount of steviol and downstream products of steviol to the total amount of kaurenoic acid, steviol, and downstream products of steviol in the resulting composition.

In certain embodiments, provided herein are host cells comprising a kaurenoic acid hydroxy lase polypeptide comprising an amino acid sequence described herein and capable of converting kaurenoic acid to steviol. In certain embodiments, provided herein are host cells comprising a kaurenoic acid hydroxy lase polypeptide comprising an amino acid sequence described herein and capable of oxidation of the 13 position of kaurenoic acid. In certain embodiments, provided herein are host cells comprising a kaurenoic acid hydroxylase polypeptide capable of converting kaurenoic acid to steviol at an efficiency greater than 30%, 35%, 40%, 45%, 50%, 55%, 60%, 65%, 70%, 75%, 80%, 85%, 90%, 95%, 96%, or 97%, and wherein the kaurenoic acid hydroxylase polypeptide comprises an amino acid sequence having at least 95% sequence identity to the amino acid sequence of SEQ ID NO: 1.

In embodiments described herein, any suitable method can be used to determine corresponding amino acid positions or corresponding loop locations of two polypeptides. In certain embodiments, the sequences of a kaurenoic acid hydroxylase polypeptide and the reference sequence SEQ ID NO: 1 can be aligned using Clustal (W or Omega) using its default parameters. In other embodiment, the sequences of a kaurenoic acid hydroxylase polypeptide and the reference sequence SEQ ID NO: 1 can be aligned using structural alignments such as SWISS-MODEL, which is a protein structure homology-modelling server, accessible via the ExPASy web server, or from the program DeepView (Swiss Pdb-Viewer).

While the Stevia rebaudiana kaurenoic acid hydroxylase polypeptide or any variant Stevia rebaudiana kaurenoic acid hydroxylase polypeptide of the host cells accepts kaurenoic acid as a substrate, the source of kaurenoic acid can be any source deemed suitable to those of skill. In certain embodiments, the Stevia rebaudiana kaurenoic acid hydroxylase polypeptide or any variant Stevia rebaudiana kaurenoic acid hydroxylase polypeptide can be contacted with kaurenoic acid. In certain embodiments, the Stevia rebaudiana kaurenoic acid hydroxylase polypeptide or any variant of Stevia rebaudiana kaurenoic acid hydroxylase polypeptide can be contacted with a composition comprising kaurenoic acid. In certain embodiments, the composition is derived or sourced from natural products isolated from Stevia rebaudiana leaves. In certain embodiments, the composition is microbially derived or sourced. In certain embodiments, the host cell can be contacted with a composition comprising one or more carbon sources.

In certain embodiments, any Stevia rebaudiana kaurenoic acid hydroxylase polypeptide suitable for catalyzing a desired reaction can be screened with any suitable method known in the art. For example, a suitable variant Stevia rebaudiana kaurenoic acid hydroxy lase polypeptide can be assayed in vivo by expressing a heterologous nucleic acid encoding a variant Stevia rebaudiana kaurenoic acid hydroxylase polypeptide and screening cells that produce functional Stevia rebaudiana kaurenoic acid hydroxylase polypeptide capable of catalyzing oxidation at a desired location of a substrate (e.g., C-13 position of kaurenoic acid). Exemplary screening methods are described in the Examples below. In another example, a suitable variant Stevia rebaudiana kaurenoic acid hydroxylase polypeptide can be screened in vitro by contacting a variant Stevia rebaudiana kaurenoic acid hydroxy lase polypeptide with a substrate such as kaurenoic acid. In this example, assaying the presence of steviol or a steviol glycoside such as RebD can be used as a test to determine whether a Stevia rebaudiana kaurenoic acid hydroxylase polypeptide is a suitable enzyme. The reaction can be analyzed by LC-MS or other known methods in the art. See, e.g. WO 2013/022989.

In certain embodiments, a variant Stevia rebaudiana kaurenoic acid hydroxylase polypeptide is considered suitable in converting kaurenoic acid to steviol if it is capable of converting kaurenoic acid to steviol at an efficiency of greater than 30%, 40%, 50%, 60%, 70%, 80%, 90%, 95%, 96%, or 97% in vivo.

In advantageous embodiments, the host cell can comprise one or more enzymatic pathways capable of making kaurenoic acid, said pathways taken individually or together. As described herein, the host cells comprise a Stevia rebaudiana kaurenoic acid hydroxylase provided herein, capable of converting kaurenoic acid to steviol. In certain embodiments, the host cell further comprises one or more enzymes capable of converting farnesyl diphosphate to geranylgeranyl diphosphate. In certain embodiments, the host cell further comprises one or more enzymes capable of converting geranylgeranyl diphosphate to copalyl diphosphate. In certain embodiments, the host cell further comprises one or more enzymes capable of converting copalyl diphosphate to kaurene. In certain embodiments, the host cell further comprises one or more enzymes capable of converting kaurene to kaurenoic acid. In certain embodiments, the host cell further comprises one or more enzymes capable of converting steviol to one or more steviol glycosides. In certain embodiments, the host cell further comprises one, two, three, four, or more enzymes together capable of converting steviol to RebA. In certain embodiments, the host cell further comprises one or more enzymes capable of converting RebA to RebD. In certain embodiments, the host cell further comprises one or more enzymes capable of converting RebD to RebM. Useful enzymes and nucleic acids encoding the enzymes are known to those of skill. Particularly useful enzymes and nucleic acids are described in the sections below and further described, for example, in US 2014/0329281 A1, US 2014/0357588 A1, US 2015/0159188, WO 2016/038095 A2, and US 2016/0198748 A1.

In further embodiments, the host cells further comprise one or more enzymes capable of making geranylgeranyl diphosphate from a carbon source. These include enzymes of the DXP pathway and enzymes of the MEV pathway. Useful enzymes and nucleic acids encoding the enzymes are known to those of skill in the art. Exemplary enzymes of each pathway are described below and further described, for example, in US 2016/0177341 A1.

In some embodiments, the host cells comprise one or more or all of the isoprenoid pathway enzymes selected from the group consisting of: (a) an enzyme that condenses two molecules of acetyl-coenzyme A to form acetoacetyl-CoA (e.g., an acetyl-coA thiolase); (b) an enzyme that condenses acetoacetyl-CoA with another molecule of acetyl-CoA to form 3-hydroxy-3-methylglutaryl-CoA (HMG-COA) (e.g., an HMG-COA synthase); (c) an enzyme that converts HMG-COA into mevalonate (e.g., an HMG-COA reductase); (d) an enzyme that converts mevalonate into mevalonate 5-phosphate (e.g., a mevalonate kinase); (e) an enzyme that converts mevalonate 5-phosphate into mevalonate 5-pyrophosphate (e.g., a phosphomevalonate kinase); (f) an enzyme that converts mevalonate 5-pyrophosphate into isopentenyl diphosphate (IPP) (e.g., a mevalonate pyrophosphate decarboxylase); (g) an enzyme that converts IPP into dimethylallyl pyrophosphate (DMAPP) (e.g., an IPP isomerase); (h) a polyprenyl synthase that can condense IPP and/or DMAPP molecules to form polyprenyl compounds containing more than five carbons; (i) an enzyme that condenses IPP with DMAPP to form geranyl pyrophosphate (GPP) (e.g., a GPP synthase); (j) an enzyme that condenses two molecules of IPP with one molecule of DMAPP (e.g., an FPP synthase); (k) an enzyme that condenses IPP with GPP to form farnesyl pyrophosphate (FPP) (e.g., an FPP synthase); (l) an enzyme that condenses IPP and DMAPP to form geranylgeranyl pyrophosphate (GGPP); and (m) an enzyme that condenses IPP and FPP to form GGPP.

In certain embodiments, the additional enzymes are native. In advantageous embodiments, the additional enzymes are heterologous. In certain embodiments, two or more enzymes can be combined in one polypeptide.

### 5.3 Cell Strains

Host cells useful compositions and methods provided herein include archae, prokaryotic, or eukaryotic cells.

Suitable prokaryotic hosts include, but are not limited, to any of a variety of gram-positive, gram-negative, or gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Streptomyces, Synechococcus, and Zymomonas. Examples of prokaryotic strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. In a particular embodiment, the host cell is an Escherichia coli cell.

Suitable archae hosts include, but are not limited to, cells belonging to the genera: Aeropyrum, Archaeglobus, Halobacterium, Methanococcus, Methanobacterium, Pyrococcus, Sulfolobus, and Thermoplasma. Examples of archae strains include, but are not limited to: Archaeoglobus fulgidus, Halobacterium sp., Methanococcus jannaschii, Methanobacterium thermoautotrophicum, Thermoplasma acidophilum, Thermoplasma volcanium, Pyrococcus horikoshii, Pyrococcus abyssi, and Aeropyrum pernix.

Suitable eukaryotic hosts include, but are not limited to, fungal cells, algal cells, insect cells, and plant cells. In some embodiments, yeasts useful in the present methods include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the host microbe is Saccharomyces cerevisiae, Pichia pastoris, Schizosaccharomyces pombe, Dekkera bruxellesis, Kluyveromyces lactis (previously called Saccharomyces lactis), Kluveromyces marxianus, Arxula adeninivorans, or Hansenula polymorpha (now known as Pichia angusta). In some embodiments, the host microbe is a strain of the genus Candida, such as Candida lipolytica, Candida guilliermondii, Candida krusei, Candida pseudotropicalis, or Candida utilis.

In a particular embodiment, the host microbe is Saccharomyces cerevisiae. In some embodiments, the host is a strain of Saccharomyces cerevisiae selected from the group consisting of Baker's yeast, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the host microbe is a strain of Saccharomyces cerevisiae selected from the group consisting of PE-2, CAT-1, VR-1, BG-1, CR-1, and SA-1. In a particular embodiment, the strain of Saccharomyces cerevisiae is PE-2. In another particular embodiment, the strain of Saccharomyces cerevisiae is CAT-1. In another particular embodiment, the strain of Saccharomyces cerevisiae is BG-1.

In some embodiments, the host microbe is a microbe that is suitable for industrial fermentation. In particular embodiments, the microbe is conditioned to subsist under high solvent concentration, high temperature, expanded substrate utilization, nutrient limitation, osmotic stress due to sugar and salts, acidity, sulfite and bacterial contamination, or combinations thereof, which are recognized stress conditions of the industrial fermentation environment.

### 5.4 The Steviol and Steviol Glycoside Biosynthesis Pathways

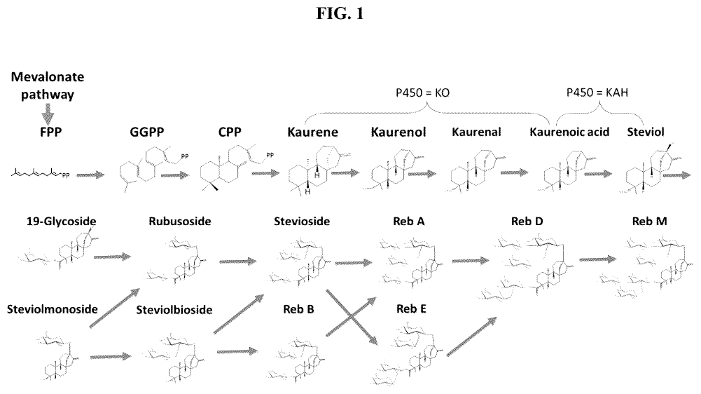

In some embodiments, a steviol biosynthesis pathway and/or a steviol glycoside biosynthesis pathway is activated in the genetically modified host cells provided herein by engineering the cells to express polynucleotides and/or polypeptides encoding one or more enzymes of the pathway. FIG. 1 illustrates an exemplary steviol biosynthesis pathway.

Thus, in some embodiments, the genetically modified host cells provided herein comprise a heterologous polynucleotide encoding a polypeptide having geranylgeranyl diphosphate synthase (GGPPS) activity. In some embodiments, the genetically modified host cells provided herein comprise a heterologous polynucleotide encoding a polypeptide having copalyl diphosphate synthase or ent-copalyl pyrophosphate synthase (CDPS; also referred to as ent-copalyl pyrophosphate synthase or CPS) activity. In some embodiments, the genetically modified host cells provided herein comprise a heterologous polynucleotide encoding a polypeptide having kaurene synthase (KS; also referred to as ent-kaurene synthase) activity. In particular embodiments, the genetically modified host cells provided herein comprise a heterologous polynucleotide encoding a polypeptide having kaurene oxidase activity (KO; also referred to as ent-kaurene 19-oxidase) as described herein. In particular embodiments, the genetically modified host cells provided herein comprise a heterologous polynucleotide encoding a polypeptide having kaurenoic acid hydroxylase polypeptide activity (KAH; also referred to as steviol synthase) according to the embodiments provided herein. In some embodiments, the genetically modified host cells provided herein comprise a heterologous polynucleotide encoding a polypeptide having cytochrome P450 reductase (CPR) activity.

In some embodiments, the genetically modified host cells provided herein comprise a heterologous polynucleotide encoding a polypeptide having UGT74G1 activity. In some embodiments, the genetically modified host cells provided herein comprise a heterologous polynucleotide encoding a polypeptide having UGT76G1 activity. In some embodiments, the genetically modified host cells provided herein comprise a heterologous polynucleotide encoding a polypeptide having UGT85C2 activity. In some embodiments, the genetically modified host cells provided herein comprise a heterologous polynucleotide encoding a polypeptide having UGT91D activity. In some embodiments, the genetically modified host cells provided herein comprise a heterologous polynucleotide encoding a polypeptide having UGT_{AD} activity. As described below, UGT_{AD} refers to a uridine diphosphate-dependent glycosyl transferase capable of transferring a glucose moiety to the C-2′ position of the 19-O-glucose of RebA to produce RebD.

In certain embodiments, the host cell comprises a variant enzyme. In certain embodiments, the variant can comprise up to 15, 10, 9, 8, 7, 6, 5, 4, 3, 2, or 1 amino acid substitutions relative to the relevant polypeptide. In certain embodiments, the variant can comprise up to 15, 10, 9, 8, 7, 6, 5, 4, 3, 2, or 1 conservative amino acid substitutions relative to the reference polypeptide. In certain embodiments, any of the nucleic acids described herein can be optimized for the host cell, for instance codon optimized.

Exemplary nucleic acids and enzymes of a steviol biosynthesis pathway and/or a steviol glycoside biosynthesis pathway are described below.

### 5.4.1 Geranylgeranyl Diphosphate Synthase (GGPPS)

Geranylgeranyl diphosphate synthases (EC 2.5.1.29) catalyze the conversion of farnesyl pyrophosphate into geranylgeranyl diphosphate. Illustrative examples of enzymes include those of Stevia rebaudiana (accession no. ABD92926), Gibberella fujikuroi (accession no. CAA75568), Mus musculus (accession no. AAH69913), Thalassiosira pseudonana (accession no. XP_002288339), Streptomyces clavuligerus (accession no. ZP_05004570), Sulfolobus acidocaldarius (accession no. BAA43200), Synechococcus sp. (accession no. ABC98596), Arabidopsis thaliana (accession no. NP_195399), Blakeslea trispora (accession no. AFC92798.1) and US 2014/0329281 A1. Nucleic acids encoding these enzymes are useful in the cells and methods provided herein. In certain embodiments, provided herein are cells and methods using a nucleic acid having at least 80%, 85%, 90%, or 95% sequence identity to at least one of these GGPPS nucleic acids. In certain embodiments, provided herein are cells and methods using a nucleic acid that encodes a polypeptide having at least 80%, 85%, 90%, 95% sequence identity to at least one of these GGPPS enzymes.

### 5.4.2 Copalyl Diphosphate Synthase (CDPS)

Copalyl diphosphate synthases (EC 5.5.1.13) catalyze the conversion of geranylgeranyl diphosphate into copalyl diphosphate. Illustrative examples of enzymes include those of Stevia rebaudiana (accession no. AAB87091), Streptomyces clavuligerus (accession no. EDY51667), Bradyrhizobium japonicum (accession no. AAC28895.1), Zea mays (accession no. AY562490), Arabidopsis thaliana (accession no. NM_116512), Oryza sativa (accession no. Q5MQ85.1) and US 2014/0329281 A1. Nucleic acids encoding these enzymes are useful in the cells and methods provided herein. In certain embodiments, provided herein are cells and methods using a nucleic acid having at least 80%, 85%, 90%, or 95% sequence identity to at least one of these CDPS nucleic acids. In certain embodiments, provided herein are cells and methods using a nucleic acid that encodes a polypeptide having at least 80%, 95%, 90%, or 95% sequence identity to at least one of these CDPS enzymes.

### 5.4.3 Kaurene Synthase (KS)

Kaurene synthases (EC 4.2.3.19) catalyze the conversion of copalyl diphosphate into kaurene and diphosphate. Illustrative examples of enzymes include those of Bradyrhizobium japonicum (accession no. AAC28895.1), Phaeosphaeria sp. (accession no. O13284), Arabidopsis thaliana (accession no. Q9SAK2), Picea glauca (accession no. ADB55711.1) and US 2014/0329281 A1. Nucleic acids encoding these enzymes are useful in the cells and methods provided herein. In certain embodiments, provided herein are cells and methods using a nucleic acid having at least 80%, 85%, 90%, or 95% sequence identity to at least one of these KS nucleic acids. In certain embodiments, provided herein are cells and methods using a nucleic acid that encodes a polypeptide having at least 80%, 85%, 85%, 90%, or 95% sequence identity to at least one of these KS enzymes.

### 5.4.4 Bifunctional Copalyl Diphosphate Synthase (CDPS) and Kaurene Synthase (KS)

CDPS-KS bifunctional enzymes (EC 5.5.1.13 and EC 4.2.3.19) also can be used. Illustrative examples of enzymes include those of Phomopsis amygdali (accession no. BAG30962), Physcomitrella patens (accession no. BAF61135), Gibberella fujikuroi (accession no. Q9UVY5.1), and US 2014/0329281 A1, US 2014/0357588 A1, US 2015/0159188, and WO 2016/038095 A2. Nucleic acids encoding these enzymes are useful in the cells and methods provided herein. In certain embodiments, provided herein are cells and methods using a nucleic acid having at least 80%, 85%, 90%, or 95% sequence identity to at least one of these CDPS-KS nucleic acids. In certain embodiments, provided herein are cells and methods using a nucleic acid that encodes a polypeptide having at least 80%, 85%, 90%, or 95% sequence identity to at least one of these CDPS-KS enzymes.

### 5.4.5 Ent-Kaurene Oxidase (KO)

Ent-kaurene oxidases (EC 1.14.13.78; also referred to as kaurene oxidases herein) catalyze the conversion of kaurene into kaurenoic acid. Illustrative examples of enzymes include those of Oryza sativa (accession no. Q5Z5R4), Gibberella fujikuroi (accession no. O94142), Arabidopsis thaliana (accession no. Q93ZB2), Stevia rebaudiana (accession no. AAQ63464.1), Pisum sativum (Uniprot no. Q6XAF4) and US 2014/0329281 A1, US 2014/0357588 A1, US 2015/0159188, and WO 2016/038095 A2. Nucleic acids encoding these enzymes are useful in the cells and methods provided herein. In certain embodiments, provided herein are cells and methods using a nucleic acid having at least 80%, 85%, 90%, or 95% sequence identity to at least one of these KO nucleic acids. In certain embodiments, provided herein are cells and methods using a nucleic acid that encodes a polypeptide having at least 80%, 85%, 90%, or 95% sequence identity to at least one of these KO enzymes.

### 5.4.6 Steviol Synthase (KAH)

Modified steviol synthases, or kaurenoic acid hydroxylases (KAH), (EC 1.14.13.79) are provided herein. Nucleic acids encoding these enzymes are useful in the cells and methods provided herein.

### 5.4.7 Cytochrome P450 Reductase (CPR)

Cytochrome P450 reductases (EC 1.6.2.4) are necessary for the activity of KO and/or KAH above. Illustrative examples of enzymes include those of Stevia rebaudiana (accession no. ABB88839) Arabidopsis thaliana (accession no. NP_194183), Gibberella fujikuroi (accession no. CAE09055), Artemisia annua (accession no. ABC47946.1) and US 2014/0329281 A1, US 2014/0357588 A1, US 2015/0159188, and WO 2016/038095 A2. Nucleic acids encoding these enzymes are useful in the cells and methods provided herein. In certain embodiments, provided herein are cells and methods using a nucleic acid having at least 80%, 85%, 90%, or 95% sequence identity to at least one of these CPR nucleic acids. In certain embodiments, provided herein are cells and methods using a nucleic acid that encodes a polypeptide having at least 80%, 85%, 90%, or 95% sequence identity to at least one of these CPR enzymes.

### 5.4.8 UDP Glycosyltransferase 74G1 (UGT74G1)

A UGT74G1 is capable of functioning as a uridine 5′-diphospho glucosyl:steviol 19-COOH transferase and as a uridine 5′-diphospho glucosyl:steviol-13-O-glucoside 19-COOH transferase. As shown in FIG. 1, a UGT74G1 is capable of converting steviol to 19-glycoside. A UGT74G1 is also capable of converting steviolmonoside to rubusoside. A UGT74G1 may be also capable of converting steviolbioside to stevioside. Illustrative examples of enzymes include those of Stevia rebaudiana (e.g., those of Richman et al., 2005, Plant J. 41: 56-67 and US 2014/0329281 and WO 2016/038095 A2 and accession no. AAR06920.1). Nucleic acids encoding these enzymes are useful in the cells and methods provided herein. In certain embodiments, provided herein are cells and methods using a nucleic acid having at least 80%, 85%, 90%, or 95% sequence identity to at least one of these UGT74G1 nucleic acids. In certain embodiments, provided herein are cells and methods using a nucleic acid that encodes a polypeptide having at least 80%, 85%, 90%, or 95% sequence identity to at least one of these UGT74G1 enzymes.

### 5.4.9 UDP Glycosyltransferase 76G1 (UGT76G1)

A UGT76G1 is capable of transferring a glucose moiety to the C-3′ of the C-13-O-glucose of the acceptor molecule, a steviol 1,2 glycoside. Thus, a UGT76G1 is capable of functioning as a uridine 5′-diphospho glucosyl:steviol 13-O-1,2 glucoside C-3′ glucosyl transferase and a uridine 5′-diphospho glucosyl:steviol-19-O-glucose, 13-O-1,2 bioside C-3′ glucosyl transferase. UGT76G1 is capable of converting steviolbioside to RebB. A UGT76G1 is also capable of converting stevioside to RebA. A UGT76G1 is also capable of converting RebD to RebM. Illustrative examples of enzymes include those of Stevia rebaudiana (e.g., those of Richman et al., 2005, Plant J. 41: 56-67 and US 2014/0329281 A1 and WO 2016/038095 A2 and accession no. AAR06912.1). Nucleic acids encoding these enzymes are useful in the cells and methods provided herein. In certain embodiments, provided herein are cells and methods using a nucleic acid having at least 80%, 85%, 90%, or 95% sequence identity to at least one of these UGT76G1 nucleic acids. In certain embodiments, provided herein are cells and methods using a nucleic acid that encodes a polypeptide having at least 80%, 85%, 90%, or 95% sequence identity to at least one of these UGT76G1 enzymes.

### 5.4.10 UDP Glycosyltransferase 85C2 (UGT85C2)

A UGT85C2 is capable of functioning as a uridine 5′-diphospho glucosyl:steviol 13-OH transferase, and a uridine 5′-diphospho glucosyl:steviol-19-O-glucoside 13-OH transferase. A UGT85C2 is capable of converting steviol to steviolmonoside, and is also capable of converting 19-glycoside to rubusoside. Illustrative examples of enzymes include those of Stevia rebaudiana (e.g., those of Richman et al., 2005, Plant J. 41: 56-67 and US 2014/0329281 A1 and WO 2016/038095 A2 and accession no. AAR06916.1). Nucleic acids encoding these enzymes are useful in the cells and methods provided herein. In certain embodiments, provided herein are cells and methods using a nucleic acid having at least 80%, 85%, 90%, or 95% sequence identity to at least one of these UGT85C2 nucleic acids. In certain embodiments, provided herein are cells and methods using a nucleic acid that encodes a polypeptide having at least 80%, 85%, 90%, or 95% sequence identity to at least one of these UGT85C2 enzymes.

### 5.4.11 UDP-Glycosyltransferase 91D (UGT91D)

A UGT91D is capable of functioning as a uridine 5′-diphosphoglucosyl:steviol-13-O-glucoside transferase, transferring a glucose moiety to the C-2′ of the 13-O-glucose of the acceptor molecule, steviol-13-O-glucoside (steviolmonoside) to produce steviolbioside. A UGT91D is also capable of functioning as a uridine 5′-diphosphoglucosyl:rubusoside transferase, transferring a glucose moiety to the C-2′ of the 13-O-glucose of the acceptor molecule, rubusoside, to provide stevioside. A UGT91D is also referred to as UGT91D2, UGT91D2e, or UGT91D-like3. Illustrative examples of UGT91D enzymes include those of Stevia rebaudiana (e.g., those of UGT sequence with accession no. ACE87855.1, US 2014/0329281 A1, WO 2016/038095 A2, and SEQ ID NO:7). Nucleic acids encoding these enzymes are useful in the cells and methods provided herein. In certain embodiments, provided herein are cells and methods using a nucleic acid having at least 80%, 85%, 90%, or 95% sequence identity to at least one of these UGT91D nucleic acids. In certain embodiments, provided herein are cells and methods using a nucleic acid that encodes a polypeptide having at least 80%, 85%, 90%, or 95% sequence identity to at least one of these UGT9ID enzymes.

### 5.4.12 Uridine Diphosphate-Dependent Glycosyl Transferase Capable of Converting RebA to RebD (UGT_{AD})

A uridine diphosphate-dependent glycosyl transferase (UGT_{AD}) is capable of transferring a glucose moiety to the C-2′ position of the 19-O-glucose of RebA to produce RebD. A UGT_{AD} is also capable of transferring a glucose moiety to the C-2′ position of the 19-O-glucose of stevioside to produce RebE. Useful examples of UGTs include Os_UGT_91C1 from Oryza sativa (also referred to as EUGT11 in Houghton-Larsen et al., WO 2013/022989 A2; XP_015629141.1) and S1_UGT_101249881 from Solanum lycopersicum (also referred to as UGTSL2 in Markosyan et al., WO2014/193888 A1; XP_004250485.1). Further useful UGTs include UGT40087 (XP_004982059.1; as described in WO 2018/031955), sr.UGT_9252778, Bd_UGT10840 (XP_003560669.1), Hv_UGT_V1 (BAJ94055.1), Bd_UGT10850 (XP_010230871.1), and Ob_UGT91B1_like (XP_006650455.1). Any UGT or UGT variant can be used in the compositions and methods described herein. Nucleic acids encoding these enzymes are useful in the cells and methods provided herein. In certain embodiments, provided herein are cells and methods using a nucleic acid having at least 80%, 85%, 90%, or 95% sequence identity to at least one of the UGTs. In certain embodiments, provided herein are cells and methods using a nucleic acid that encodes a polypeptide having at least 80%, 85%, 90%, or 95% sequence identity to at least one of these UGTs. In certain embodiments, provided herein are a nucleic acid that encodes a UGT variant described herein.

### 5.5 MEV Pathway FPP and/or GGPP Production

In some embodiments, a genetically modified host cell provided herein comprises one or more heterologous enzymes of the MEV pathway, useful for the formation of FPP and/or GGPP. In some embodiments, the one or more enzymes of the MEV pathway comprise an enzyme that condenses acetyl-CoA with malonyl-CoA to form acetoacetyl-CoA. In some embodiments, the one or more enzymes of the MEV pathway comprise an enzyme that condenses two molecules of acetyl-CoA to form acetoacetyl-CoA. In some embodiments, the one or more enzymes of the MEV pathway comprise an enzyme that condenses acetoacetyl-CoA with acetyl-CoA to form HMG-COA. In some embodiments, the one or more enzymes of the MEV pathway comprise an enzyme that converts HMG-COA to mevalonate. In some embodiments, the one or more enzymes of the MEV pathway comprise an enzyme that phosphorylates mevalonate to mevalonate 5-phosphate. In some embodiments, the one or more enzymes of the MEV pathway comprise an enzyme that converts mevalonate 5-phosphate to mevalonate 5-pyrophosphate. In some embodiments, the one or more enzymes of the MEV pathway comprise an enzyme that converts mevalonate 5-pyrophosphate to isopentenyl pyrophosphate. In some embodiments, the one or more enzymes of the MEV pathway comprise an enzyme that converts isopentenyl pyrophosphate to dimethylallyl diphosphate.

In some embodiments, the one or more enzymes of the MEV pathway are selected from the group consisting of acetyl-CoA thiolase, acetoacetyl-CoA synthetase, HMG-COA synthase, HMG-CoA reductase, mevalonate kinase, phosphomevalonate kinase, mevalonate pyrophosphate decarboxylase, and isopentyl diphosphate:dimethylallyl diphosphate isomerase (IDI or IPP isomerase). In some embodiments, with regard to the enzyme of the MEV pathway capable of catalyzing the formation of acetoacetyl-CoA, the genetically modified host cell comprises either an enzyme that condenses two molecules of acetyl-CoA to form acetoacetyl-CoA, e.g., acetyl-CoA thiolase; or an enzyme that condenses acetyl-CoA with malonyl-CoA to form acetoacetyl-CoA, e.g., acetoacetyl-CoA synthase. In some embodiments, the genetically modified host cell comprises both an enzyme that condenses two molecules of acetyl-CoA to form acetoacetyl-CoA, e.g., acetyl-CoA thiolase; and an enzyme that condenses acetyl-CoA with malonyl-CoA to form acetoacetyl-CoA, e.g., acetoacetyl-CoA synthase.

In some embodiments, the host cell comprises one or more heterologous nucleotide sequences encoding more than one enzyme of the MEV pathway. In some embodiments, the host cell comprises one or more heterologous nucleotide sequences encoding two enzymes of the MEV pathway. In some embodiments, the host cell comprises one or more heterologous nucleotide sequences encoding an enzyme that can convert HMG-CoA into mevalonate and an enzyme that can convert mevalonate into mevalonate 5-phosphate. In some embodiments, the host cell comprises one or more heterologous nucleotide sequences encoding three enzymes of the MEV pathway. In some embodiments, the host cell comprises one or more heterologous nucleotide sequences encoding four enzymes of the MEV pathway. In some embodiments, the host cell comprises one or more heterologous nucleotide sequences encoding five enzymes of the MEV pathway. In some embodiments, the host cell comprises one or more heterologous nucleotide sequences encoding six enzymes of the MEV pathway. In some embodiments, the host cell comprises one or more heterologous nucleotide sequences encoding seven enzymes of the MEV pathway. In some embodiments, the host cell comprises a plurality of heterologous nucleic acids encoding all of the enzymes of the MEV pathway.

In some embodiments, the genetically modified host cell further comprises a heterologous nucleic acid encoding an enzyme that can convert isopentenyl pyrophosphate (IPP) into dimethylallyl pyrophosphate (DMAPP). In some embodiments, the genetically modified host cell further comprises a heterologous nucleic acid encoding an enzyme that can condense IPP and/or DMAPP molecules to form a polyprenyl compound. In some embodiments, the genetically modified host cell further comprises a heterologous nucleic acid encoding an enzyme that can modify IPP or a polyprenyl to form an isoprenoid compound such as FPP.

### 5.5.1 Conversion of Acetyl-CoA to Acetoacetyl-CoA

In some embodiments, the genetically modified host cell comprises a heterologous nucleotide sequence encoding an enzyme that can condense two molecules of acetyl-coenzyme A to form acetoacetyl-CoA, e.g., an acetyl-CoA thiolase. Illustrative examples of nucleotide sequences encoding such an enzyme include but are not limited to: (NC_000913 REGION: 2324131.2325315; Escherichia coli), (D49362; Paracoccus denitrificans), and (L20428; Saccharomyces cerevisiae).

Acetyl-CoA thiolase catalyzes the reversible condensation of two molecules of acetyl-CoA to yield acetoacetyl-CoA, but this reaction is thermodynamically unfavorable; acetoacetyl-CoA thiolysis is favored over acetoacetyl-CoA synthesis. Acetoacetyl-CoA synthase (AACS) (alternately referred to as acetyl-CoA:malonyl-CoA acyltransferase; EC 2.3.1.194) condenses acetyl-CoA with malonyl-CoA to form acetoacetyl-CoA. In contrast to acetyl-CoA thiolase, AACS-catalyzed acetoacetyl-CoA synthesis is essentially an energy-favored reaction, due to the associated decarboxylation of malonyl-CoA. In addition, AACS exhibits no thiolysis activity against acetoacetyl-CoA, and thus the reaction is irreversible.

In host cells comprising acetyl-CoA thiolase and a heterologous ADA and/or phosphotransacetylase (PTA), the reversible reaction catalyzed by acetyl-CoA thiolase, which favors acetoacetyl-CoA thiolysis, may result in a large acetyl-CoA pool. In view of the reversible activity of ADA, this acetyl-CoA pool may in turn drive ADA towards the reverse reaction of converting acetyl-CoA to acetaldehyde, thereby diminishing the benefits provided by ADA towards acetyl-CoA production. Similarly, the activity of PTA is reversible, and thus, a large acetyl-CoA pool may drive PTA towards the reverse reaction of converting acetyl-CoA to acetyl phosphate. Therefore, in some embodiments, in order to provide a strong pull on acetyl-CoA to drive the forward reaction of ADA and PTA, the MEV pathway of the genetically modified host cell provided herein utilizes an acetoacetyl-CoA synthase to form acetoacetyl-CoA from acetyl-CoA and malonyl-CoA.

In some embodiments, the AACS is from Streptomyces sp. strain CL 190 (Okamura et al., Proc Natl Acad Sci USA 107(25): 11265-70 (2010). Representative AACS nucleotide sequences of Streptomyces sp. strain CL190 include accession number AB540131.1. Representative AACS protein sequences of Streptomyces sp. strain CL190 include accession numbers D7URV0, BAJ10048. Other acetoacetyl-CoA synthases useful for the compositions and methods provided herein include, but are not limited to, Streptomyces sp. (AB183750; KO-3988 BAD86806); S. anulatus strain 9663 (FN178498; CAX48662); Streptomyces sp. KO-3988 (AB212624; BAE78983); Actinoplanes sp. A40644 (AB113568; BAD07381); Streptomyces sp. C (NZ_ACEW010000640; ZP_05511702); Nocardiopsis dassonvillei DSM 43111 (NZ_ABUI01000023; ZP_04335288); Mycobacterium ulcerans Agy99 (NC_008611; YP_907152); Mycobacterium marinum M (NC_010612; YP_001851502); Streptomyces sp. Mg1 (NZ_DS570501; ZP_05002626); Streptomyces sp. AA4 (NZ_ACEV01000037; ZP_05478992); S. roseosporus NRRL 15998 (NZ_ABYB01000295; ZP_04696763); Streptomyces sp. ACTE (NZ_ADFD01000030; ZP_06275834); S. viridochromogenes DSM 40736 (NZ_ACEZ01000031; ZP_05529691); Frankia sp. CcI3 (NC_007777; YP_480101); Nocardia brasiliensis (NC_018681; YP_006812440.1); and Austwickia chelonae (NZ_BAGZ01000005; ZP_10950493.1). Additional suitable acetoacetyl-CoA synthases include those described in U.S. Patent Application Publication Nos. 2010/0285549 and 2011/0281315, the contents of which are incorporated by reference in their entireties.

Acetoacetyl-CoA synthases also useful in the compositions and methods provided herein include those molecules which are said to be “derivatives” of any of the acetoacetyl-CoA synthases described herein. Such a “derivative” has the following characteristics: (1) it shares substantial homology with any of the acetoacetyl-CoA synthases described herein; and (2) is capable of catalyzing the irreversible condensation of acetyl-CoA with malonyl-CoA to form acetoacetyl-CoA. A derivative of an acetoacetyl-CoA synthase is said to share “substantial homology” with acetoacetyl-CoA synthase if the amino acid sequences of the derivative is at least 80%, and more preferably at least 90%, and most preferably at least 95%, the same as that of acetoacetyl-CoA synthase.

### 5.5.2 Conversion of Acetoacetyl-CoA to HMG-COA

In some embodiments, the host cell comprises a heterologous nucleotide sequence encoding an enzyme that can condense acetoacetyl-CoA with another molecule of acetyl-CoA to form 3-hydroxy-3-methylglutaryl-CoA (HMG-COA), e.g., an HMG-COA synthase. Illustrative examples of nucleotide sequences encoding such an enzyme include but are not limited to: (NC_001145. complement 19061.20536; Saccharomyces cerevisiae), (X96617; Saccharomyces cerevisiae), (X83882; Arabidopsis thaliana), (AB037907; Kitasatospora griseola), (BT007302; Homo sapiens), and (NC_002758, Locus tag SAV2546, GeneID 1122571; Staphylococcus aureus).

### 5.5.3 Conversion of HMG-COA to Mevalonate

In some embodiments, the host cell comprises a heterologous nucleotide sequence encoding an enzyme that can convert HMG-COA into mevalonate, e.g., an HMG-COA reductase. In some embodiments, HMG-COA reductase is an NADH-using hydroxymethylglutaryl-CoA reductase-CoA reductase. HMG-COA reductases (EC 1.1.1.34; EC 1.1.1.88) catalyze the reductive deacylation of(S)-HMG-COA to (R)-mevalonate, and can be categorized into two classes, class I and class II HMGrs. Class I includes the enzymes from eukaryotes and most archaea, and class II includes the HMG-COA reductases of certain prokaryotes and archaea. In addition to the divergence in the sequences, the enzymes of the two classes also differ with regard to their cofactor specificity. Unlike the class I enzymes, which utilize NADPH exclusively, the class II HMG-COA reductases vary in the ability to discriminate between NADPH and NADH. See, e.g., Hedl et al., Journal of Bacteriology 186 (7): 1927-1932 (2004). Co-factor specificities for select class II HMG-COA reductases are provided below.

### Co-Factor Specificities for Select Class II HMG-COA Reductases

| | Coenzyme | | |
|---|---|---|---|
| Source | specificity | Kₘ^{NADPH} (μM) | Kₘ^{NADH} (μM) |
| P. mevalonii | NADH | | 80 |
| A. fulgidus | NAD(P)H | 500 | 160 |
| S. aureus | NAD(P)H | 70 | 100 |
| E. faecalis | NADPH | 30 | |

Useful HMG-COA reductases for the compositions and methods provided herein include HMG-COA reductases that are capable of utilizing NADH as a cofactor, e.g., HMG-CoA reductase from P. mevalonii, A. fulgidus or S. aureus. In particular embodiments, the HMG-COA reductase is capable of only utilizing NADH as a cofactor, e.g., HMG-COA reductase from P. mevalonii, S. pomeroyi or D. acidovorans.

In some embodiments, the NADH-using HMG-COA reductase is from Pseudomonas mevalonii. The sequence of the wild-type mvaA gene of Pseudomonas mevalonii, which encodes HMG-COA reductase (EC 1.1.1.88), has been previously described. See Beach and Rodwell, J. Bacteriol. 171:2994-3001 (1989). Representative mvaA nucleotide sequences of Pseudomonas mevalonii include accession number M24015. Representative HMG-CoA reductase protein sequences of Pseudomonas mevalonii include accession numbers AAA25837, P13702, MVAA_PSEMV.

In some embodiments, the NADH-using HMG-COA reductase is from Silicibacter pomeroyi. Representative HMG-COA reductase nucleotide sequences of Silicibacter pomeroyi include accession number NC_006569.1. Representative HMG-COA reductase protein sequences of Silicibacter pomeroyi include accession number YP_164994.

In some embodiments, the NADH-using HMG-COA reductase is from Delftia acidovorans. A representative HMG-COA reductase nucleotide sequences of Delftia acidovorans includes NC_010002 REGION: complement (319980 . . . 321269). Representative HMG-COA reductase protein sequences of Delftia acidovorans include accession number YP_001561318.

In some embodiments, the NADH-using HMG-COA reductases is from Solanum tuberosum (Crane et al., J. Plant Physiol. 159:1301-1307 (2002)).

NADH-using HMG-COA reductases also useful in the compositions and methods provided herein include those molecules which are said to be “derivatives” of any of the NADH-using HMG-COA reductases described herein, e.g., from P. mevalonii, S. pomeroyi and D. acidovorans. Such a “derivative” has the following characteristics: (1) it shares substantial homology with any of the NADH-using HMG-COA reductases described herein; and (2) is capable of catalyzing the reductive deacylation of(S)-HMG-COA to (R)-mevalonate while preferentially using NADH as a cofactor. A derivative of an NADH-using HMG-COA reductase is said to share “substantial homology” with NADH-using HMG-COA reductase if the amino acid sequences of the derivative is at least 80%, and more preferably at least 90%, and most preferably at least 95%, the same as that of NADH-using HMG-COA reductase.

As used herein, the phrase “NADH-using” means that the NADH-using HMG-COA reductase is selective for NADH over NADPH as a cofactor, for example, by demonstrating a higher specific activity for NADH than for NADPH. In some embodiments, selectivity for NADH as a cofactor is expressed as a k_{cat}^{(NADH)}/k_{cat}^{(NADPH)} ratio. In some embodiments, the NADH-using HMG-COA reductase has a k_{cat}^{(NADH)}/k_{cat}^{(NADPH)} ratio of at least 5, 10, 15, 20, 25 or greater than 25. In some embodiments, the NADH-using HMG-COA reductase uses NADH exclusively. For example, an NADH-using HMG-COA reductase that uses NADH exclusively displays some activity with NADH supplied as the sole cofactor in vitro, and displays no detectable activity when NADPH is supplied as the sole cofactor. Any method for determining cofactor specificity known in the art can be utilized to identify HMG-COA reductases having a preference for NADH as cofactor, including those described by Kim et al., Protein Science 9:1226-1234 (2000); and Wilding et al., J. Bacteriol. 182(18):5147-52 (2000), the contents of which are hereby incorporated in their entireties.

In some embodiments, the NADH-using HMG-COA reductase is engineered to be selective for NADH over NAPDH, for example, through site-directed mutagenesis of the cofactor-binding pocket. Methods for engineering NADH-selectivity are described in Watanabe et al., Microbiology 153:3044-3054 (2007), and methods for determining the cofactor specificity of HMG-COA reductases are described in Kim et al., Protein Sci. 9:1226-1234 (2000), the contents of which are hereby incorporated by reference in their entireties.

In some embodiments, the NADH-using HMG-COA reductase is derived from a host species that natively comprises a mevalonate degradative pathway, for example, a host species that catabolizes mevalonate as its sole carbon source. Within these embodiments, the NADH-using HMG-COA reductase, which normally catalyzes the oxidative acylation of internalized (R)-mevalonate to (S)-HMG-COA within its native host cell, is utilized to catalyze the reverse reaction, that is, the reductive deacylation of (S)-HMG-COA to (R)-mevalonate, in a genetically modified host cell comprising a mevalonate biosynthetic pathway. Prokaryotes capable of growth on mevalonate as their sole carbon source have been described by: Anderson et al., J. Bacteriol. 171(12):6468-6472 (1989); Beach et al., J. Bacteriol. 171:2994-3001 (1989); Bensch et al., J. Biol. Chem. 245:3755-3762; Fimongnari et al., Biochemistry 4:2086-2090 (1965); Siddiqi et al., Biochem. Biophys. Res. Commun. 8:110-113 (1962); Siddiqi et al., J. Bacteriol. 93:207-214 (1967); and Takatsuji et al., Biochem. Biophys. Res. Commun. 110:187-193 (1983), the contents of which are hereby incorporated by reference in their entireties.

In some embodiments of the compositions and methods provided herein, the host cell comprises both a NADH-using HMGR and an NADPH-using HMG-COA reductase. Illustrative examples of nucleotide sequences encoding an NADPH-using HMG-COA reductase include, but are not limited to: (NM_206548; Drosophila melanogaster), (NC_002758, Locus tag SAV2545, GeneID 1122570; Staphylococcus aureus), (AB015627; Streptomyces sp. KO 3988), (AX128213, providing the sequence encoding a truncated HMG-CoA reductase; Saccharomyces cerevisiae), and (NC_001145: complement (115734.118898; Saccharomyces cerevisiae).

### 5.5.4 Conversion of Mevalonate to Mevalonate-5-Phosphate

In some embodiments, the host cell comprises a heterologous nucleotide sequence encoding an enzyme that can convert mevalonate into mevalonate 5-phosphate, e.g., a mevalonate kinase. Illustrative examples of nucleotide sequences encoding such an enzyme include, but are not limited to: (L77688; Arabidopsis thaliana), and (X55875; Saccharomyces cerevisiae).

### 5.5.5 Conversion of Mevalonate-5-Phosphate to Mevalonate-5-Pyrophosphate

In some embodiments, the host cell comprises a heterologous nucleotide sequence encoding an enzyme that can convert mevalonate 5-phosphate into mevalonate 5-pyrophosphate, e.g., a phosphomevalonate kinase. Illustrative examples of nucleotide sequences encoding such an enzyme include, but are not limited to: (AF429385; Hevea brasiliensis), (NM_006556; Homo sapiens), and (NC_001145. complement 712315.713670; Saccharomyces cerevisiae).

### 5.5.6 Conversion of Mevalonate-5-Pyrophosphate to IPP

In some embodiments, the host cell comprises a heterologous nucleotide sequence encoding an enzyme that can convert mevalonate 5-pyrophosphate into isopentenyl diphosphate (IPP), e.g., a mevalonate pyrophosphate decarboxylase. Illustrative examples of nucleotide sequences encoding such an enzyme include, but are not limited to: (X97557; Saccharomyces cerevisiae), (AF290095; Enterococcus faecium), and (U49260; Homo sapiens).

### 5.5.7 Conversion of IPP to DMAPP

In some embodiments, the host cell further comprises a heterologous nucleotide sequence encoding an enzyme that can convert IPP generated via the MEV pathway into dimethylallyl pyrophosphate (DMAPP), e.g., an IPP isomerase. Illustrative examples of nucleotide sequences encoding such an enzyme include, but are not limited to: (NC_000913, 3031087.3031635; Escherichia coli), and (AF082326; Haematococcus pluvialis).

### 5.5.8 Polyprenyl Synthases

In some embodiments, the host cell further comprises a heterologous nucleotide sequence encoding a polyprenyl synthase that can condense IPP and/or DMAPP molecules to form polyprenyl compounds containing more than five carbons.

In some embodiments, the host cell comprises a heterologous nucleotide sequence encoding an enzyme that can condense one molecule of IPP with one molecule of DMAPP to form one molecule of geranyl pyrophosphate (“GPP”), e.g., a GPP synthase. Illustrative examples of nucleotide sequences encoding such an enzyme include, but are not limited to: (AF513111; Abies grandis), (AF513112; Abies grandis), (AF513113; Abies grandis), (AY534686; Antirrhinum majus), (AY534687; Antirrhinum majus), (Y17376; Arabidopsis thaliana), (AE016877, Locus AP11092; Bacillus cereus; ATCC 14579), (AJ243739; Citrus sinensis), (AY534745; Clarkia breweri), (AY953508; Ips pini), (DQ286930; Lycopersicon esculentum), (AF182828; Mentha x piperita), (AF182827; Mentha x piperita), (MPI249453; Mentha x piperita), (PZE431697, Locus CAD24425; Paracoccus zeaxanthinifaciens), (AY866498; Picrorhiza kurrooa), (AY351862; Vitis vinifera), and (AF203881, Locus AAF12843; Zymomonas mobilis).

In some embodiments, the host cell comprises a heterologous nucleotide sequence encoding an enzyme that can condense two molecules of IPP with one molecule of DMAPP, or add a molecule of IPP to a molecule of GPP, to form a molecule of farnesyl pyrophosphate (“FPP”), e.g., a FPP synthase. Illustrative examples of nucleotide sequences that encode such an enzyme include, but are not limited to: (ATU80605; Arabidopsis thaliana), (ATHFPS2R; Arabidopsis thaliana), (AAU36376; Artemisia annua), (AF461050; Bos taurus), (D00694; Escherichia coli K-12), (AE009951, Locus AAL95523; Fusobacterium nucleatum subsp. nucleatum ATCC 25586), (GFFPPSGEN; Gibberella fujikuroi), (CP000009, Locus AAW60034; Gluconobacter oxydans 621H), (AF019892; Helianthus annuus), (HUMFAPS; Homo sapiens), (KLPFPSQCR; Kluyveromyces lactis), (LAU15777; Lupinus albus), (LAU20771; Lupinus albus), (AF309508; Mus musculus), (NCFPPSGEN; Neurospora crassa), (PAFPS1; Parthenium argentatum), (PAFPS2; Parthenium argentatum), (RATFAPS; Rattus norvegicus), (YSCFPP; Saccharomyces cerevisiae), (D89104; Schizosaccharomyces pombe), (CP000003, Locus AAT87386; Streptococcus pyogenes), (CP000017, Locus AAZ51849; Streptococcus pyogenes), (NC_008022, Locus YP_598856; Streptococcus pyogenes MGAS10270), (NC_008023, Locus YP_600845; Streptococcus pyogenes MGAS2096), (NC_008024, Locus YP_602832; Streptococcus pyogenes MGAS10750), (MZEFPS; Zea mays), (AE000657, Locus AAC06913; Aquifex aeolicus VF5), (NM_202836; Arabidopsis thaliana), (D84432, Locus BAA12575; Bacillus subtilis), (U12678, Locus AAC28894; Bradyrhizobium japonicum USDA 110), (BACFDPS; Geobacillus stearothermophilus), (NC_002940, Locus NP_873754; Haemophilus ducreyi 35000HP), (L42023, Locus AAC23087; Haemophilus influenzae Rd KW20), (J05262; Homo sapiens), (YP_395294; Lactobacillus sakei subsp. sakei 23K), (NC_005823, Locus YP_000273; Leptospira interrogans serovar Copenhageni str. Fiocruz L1-130), (AB003187; Micrococcus luteus), (NC_002946, Locus YP_208768; Neisseria gonorrhoeae FA 1090), (U00090, Locus AAB91752; Rhizobium sp. NGR234), (J05091; Saccharomyces cerevisiae), (CP000031, Locus AAV93568; Silicibacter pomeroyi DSS-3), (AE008481, Locus AAK99890; Streptococcus pneumoniae R6), and (NC_004556, Locus NP 779706; Xylella fastidiosa Temecula1).

In some embodiments, the host cell further comprises a heterologous nucleotide sequence encoding an enzyme that can combine IPP and DMAPP or IPP and FPP to form geranylgeranyl pyrophosphate (“GGPP”). Illustrative examples of nucleotide sequences that encode such an enzyme include, but are not limited to: (ATHGERPYRS; Arabidopsis thaliana), (BT005328; Arabidopsis thaliana), (NM_119845; Arabidopsis thaliana), (NZ_AAJM01000380, Locus ZP_00743052; Bacillus thuringiensis serovar israelensis. ATCC 35646 sq1563), (CRGGPPS; Catharanthus roseus), (NZ_AABF02000074, Locus ZP 00144509; Fusobacterium nucleatum subsp. vincentii, ATCC 49256), (GFGGPPSGN; Gibberella fujikuroi), (AY371321; Ginkgo biloba), (AB055496; Hevea brasiliensis), (AB017971; Homo sapiens), (MCI276129; Mucor circinelloides f. lusitanicus), (AB016044; Mus musculus), (AABX01000298, Locus NCU01427; Neurospora crassa), (NCU20940; Neurospora crassa), (NZ_AAKL01000008, Locus ZP_00943566; Ralstonia solanacearum UW551), (AB118238; Rattus norvegicus), (SCU31632; Saccharomyces cerevisiae), (AB016095; Synechococcus elongates), (SAGGPS; Sinapis alba), (SSOGDS; Sulfolobus acidocaldarius), (NC_007759, Locus YP_461832; Syntrophus aciditrophicus SB), (NC_006840, Locus YP_204095; Vibrio fischeri ES114), (NM_112315; Arabidopsis thaliana), (ERWCRTE; Pantoea agglomerans), (D90087, Locus BAA14124; Pantoea ananatis), (X52291, Locus CAA36538; Rhodobacter capsulatus), (AF195122, Locus AAF24294; Rhodobacter sphaeroides), and (NC_004350, Locus NP_721015; Streptococcus mutans UA159).

While examples of the enzymes of the mevalonate pathway are described above, in certain embodiments, enzymes of the DXP pathway can be used as an alternative or additional pathway to produce DMAPP and IPP in the host cells, compositions and methods described herein. Enzymes and nucleic acids encoding the enzymes of the DXP pathway are well-known and characterized in the art, e.g., WO 2012/135591 A2.

### 5.6 Methods of Producing Steviol Glycosides

In another aspect, provided herein is a method for the production of a steviol glycoside, the method comprising the steps of: (a) culturing a population of any of the genetically modified host cells described herein that are capable of producing a steviol glycoside in a medium with a carbon source under conditions suitable for making the steviol glycoside compound; and (b) recovering said steviol glycoside compound from the medium.

In some embodiments, the genetically modified host cell produces an increased amount of the steviol glycoside compared to a parent cell not comprising the one or more modifications, or a parent cell comprising only a subset of the one or more modifications of the genetically modified host cell, but is otherwise genetically identical. In some embodiments, the increased amount is at least 1%, 5%, 10%, 15%, 20%, 25%, 30%, 35%, 40%, 45%, 50%, 55%, 60%, 65%, 70%, 75%, 80%, 85%, 90%, 95%, 100% or greater than 100%, as measured, for example, in yield, production, and/or productivity, in grams per liter of cell culture, milligrams per gram of dry cell weight, on a per unit volume of cell culture basis, on a per unit dry cell weight basis, on a per unit volume of cell culture per unit time basis, or on a per unit dry cell weight per unit time basis.

In some embodiments, the host cell produces an elevated level of a steviol glycoside that is greater than about 1 gram per liter of fermentation medium. In some embodiments, the host cell produces an elevated level of a steviol glycoside that is greater than about 5 grams per liter of fermentation medium. In some embodiments, the host cell produces an elevated level of a steviol glycoside that is greater than about 10 grams per liter of fermentation medium. In some embodiments, the steviol glycoside is produced in an amount from about 10 to about 50 grams, from about 10 to about 15 grams, more than about 15 grams, more than about 20 grams, more than about 25 grams, or more than about 30 grams per liter of cell culture.

In some embodiments, the host cell produces an elevated level of a steviol glycoside that is greater than about 50 milligrams per gram of dry cell weight. In some such embodiments, the steviol glycoside is produced in an amount from about 50 to about 1500 milligrams, more than about 100 milligrams, more than about 150 milligrams, more than about 200 milligrams, more than about 250 milligrams, more than about 500 milligrams, more than about 750 milligrams, or more than about 1000 milligrams per gram of dry cell weight.

In some embodiments, the host cell produces an elevated level of a steviol glycoside that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of steviol glycoside produced by a parent cell, on a per unit volume of cell culture basis.

In some embodiments, the host cell produces an elevated level of a steviol glycoside that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of steviol glycoside produced by the parent cell, on a per unit dry cell weight basis.

In some embodiments, the host cell produces an elevated level of a steviol glycoside that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of steviol glycoside produced by the parent cell, on a per unit volume of cell culture per unit time basis.

In some embodiments, the host cell produces an elevated level of a steviol glycoside that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of steviol glycoside produced by the parent cell, on a per unit dry cell weight per unit time basis.

In most embodiments, the production of the elevated level of steviol glycoside by the host cell is inducible by the presence of an inducing compound. Such a host cell can be manipulated with ease in the absence of the inducing compound. The inducing compound is then added to induce the production of the elevated level of steviol glycoside by the host cell. In other embodiments, production of the elevated level of steviol glycoside by the host cell is inducible by changing culture conditions, such as, for example, the growth temperature, media constituents, and the like.

### 5.7 Culture Media and Conditions

Materials and methods for the maintenance and growth of microbial cultures are well known to those skilled in the art of microbiology or fermentation science (see, for example. Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the host cell, the fermentation, and the process.

The methods of producing steviol glycosides provided herein may be performed in a suitable culture medium (e.g., with or without pantothenate supplementation) in a suitable container, including but not limited to a cell culture plate, a microtiter plate, a flask, or a fermentor. Further, the methods can be performed at any scale of fermentation known in the art to support industrial production of microbial products. Any suitable fermentor may be used including a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric, et al, in Ullmann's Encyclopedia of Industrial Chemistry, Sixth Edition, Volume 12, pages 398-473, Wiley-VCH Verlag Gmbh & Co. KDaA, Weinheim, Germany.

In some embodiments, the culture medium is any culture medium in which a genetically modified microorganism capable of producing an steviol glycoside can subsist, i.e., maintain growth and viability. In some embodiments, the culture medium is an aqueous medium comprising assimilable carbon, nitrogen and phosphate sources. Such a medium can also include appropriate salts, minerals, metals and other nutrients. In some embodiments, the carbon source and each of the essential cell nutrients, are added incrementally or continuously to the fermentation media, and each required nutrient is maintained at essentially the minimum level needed for efficient assimilation by growing cells, for example, in accordance with a predetermined cell growth curve based on the metabolic or respiratory function of the cells which convert the carbon source to a biomass.

Suitable conditions and suitable media for culturing microorganisms are well known in the art. In some embodiments, the suitable medium is supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

In some embodiments, the carbon source is a monosaccharide (simple sugar), a disaccharide, a polysaccharide, a non-fermentable carbon source, or one or more combinations thereof. Non-limiting examples of suitable monosaccharides include glucose, galactose, mannose, fructose, xylose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate and glycerol.

The concentration of a carbon source, such as glucose, in the culture medium is sufficient to promote cell growth, but is not so high as to repress growth of the microorganism used. Typically, cultures are run with a carbon source, such as glucose, being added at levels to achieve the desired level of growth and biomass. In other embodiments, the concentration of a carbon source, such as glucose, in the culture medium is greater than about 1 g/L, preferably greater than about 2 g/L, and more preferably greater than about 5 g/L. In addition, the concentration of a carbon source, such as glucose, in the culture medium is typically less than about 100 g/L, preferably less than about 50 g/L, and more preferably less than about 20 g/L. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. Beyond certain concentrations, however, the addition of a nitrogen source to the culture medium is not advantageous for the growth of the microorganisms. As a result, the concentration of the nitrogen sources, in the culture medium is less than about 20 g/L, preferably less than about 10 g/L and more preferably less than about 5 g/L. Further, in some instances it may be desirable to allow the culture medium to become depleted of the nitrogen sources during culture.

The effective culture medium can contain other compounds such as inorganic salts, vitamins, trace metals or growth promoters. Such other compounds can also be present in carbon, nitrogen or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate and mixtures thereof. Typically, the concentration of phosphate in the culture medium is greater than about 1.0 g/L, preferably greater than about 2.0 g/L and more preferably greater than about 5.0 g/L. Beyond certain concentrations, however, the addition of phosphate to the culture medium is not advantageous for the growth of the microorganisms. Accordingly, the concentration of phosphate in the culture medium is typically less than about 20 g/L, preferably less than about 15 g/L and more preferably less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, preferably greater than about 1.0 g/L, and more preferably greater than about 2.0 g/L. Beyond certain concentrations, however, the addition of magnesium to the culture medium is not advantageous for the growth of the microorganisms. Accordingly, the concentration of magnesium in the culture medium is typically less than about 10 g/L, preferably less than about 5 g/L, and more preferably less than about 3 g/L. Further, in some instances it may be desirable to allow the culture medium to become depleted of a magnesium source during culture.

In some embodiments, the culture medium can also include a biologically acceptable chelating agent, such as the dihydrate of trisodium citrate. In such instance, the concentration of a chelating agent in the culture medium is greater than about 0.2 g/L, preferably greater than about 0.5 g/L, and more preferably greater than about 1 g/L. Beyond certain concentrations, however, the addition of a chelating agent to the culture medium is not advantageous for the growth of the microorganisms. Accordingly, the concentration of a chelating agent in the culture medium is typically less than about 10 g/L, preferably less than about 5 g/L, and more preferably less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid and mixtures thereof. Biologically acceptable bases include, but are not limited to, ammonium hydroxide, sodium hydroxide, potassium hydroxide and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, preferably within the range of from about 20 mg/L to about 1000 mg/L, and more preferably in the range of from about 50 mg/L to about 500 mg/L.

The culture medium can also include sodium chloride. Typically, the concentration of sodium chloride in the culture medium is within the range of from about 0.1 g/L to about 5 g/L, preferably within the range of from about 1 g/L to about 4 g/L, and more preferably in the range of from about 2 g/L to about 4 g/L.

In some embodiments, the culture medium can also include trace metals. Such trace metals can be added to the culture medium as a stock solution that, for convenience, can be prepared separately from the rest of the culture medium. Typically, the amount of such a trace metals solution added to the culture medium is greater than about 1 ml/L, preferably greater than about 5 mL/L, and more preferably greater than about 10 mL/L. Beyond certain concentrations, however, the addition of a trace metals to the culture medium is not advantageous for the growth of the microorganisms. Accordingly, the amount of such a trace metals solution added to the culture medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

In some embodiments, the culture media can include other vitamins, such as pantothenate, biotin, calcium, pantothenate, inositol, pyridoxine-HCl, and thiamine-HCl. Such vitamins can be added to the culture medium as a stock solution that, for convenience, can be prepared separately from the rest of the culture medium. Beyond certain concentrations, however, the addition of vitamins to the culture medium is not advantageous for the growth of the microorganisms.

The fermentation methods described herein can be performed in conventional culture modes, which include, but are not limited to, batch, fed-batch, cell recycle, continuous and semi-continuous. In some embodiments, the fermentation is carried out in fed-batch mode. In such a case, some of the components of the medium are depleted during culture, including pantothenate during the production stage of the fermentation. In some embodiments, the culture may be supplemented with relatively high concentrations of such components at the outset, for example, of the production stage, so that growth and/or steviol glycoside production is supported for a period of time before additions are required. The preferred ranges of these components are maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those in the art, the rate of consumption of nutrient increases during culture as the cell density of the medium increases. Moreover, to avoid introduction of foreign microorganisms into the culture medium, addition is performed using aseptic addition methods, as are known in the art. In addition, a small amount of anti-foaming agent may be added during the culture.

The temperature of the culture medium can be any temperature suitable for growth of the genetically modified cells and/or production of steviol glycoside. For example, prior to inoculation of the culture medium with an inoculum, the culture medium can be brought to and maintained at a temperature in the range of from about 20° C. to about 45° C., preferably to a temperature in the range of from about 25° C. to about 40° C., and more preferably in the range of from about 28° C. to about 32° C.

The pH of the culture medium can be controlled by the addition of acid or base to the culture medium. In such cases when ammonia is used to control pH, it also conveniently serves as a nitrogen source in the culture medium. Preferably, the pH is maintained from about 3.0 to about 8.0, more preferably from about 3.5 to about 7.0, and most preferably from about 4.0 to about 6.5.

In some embodiments, the carbon source concentration, such as the glucose concentration, of the culture medium is monitored during culture. Glucose concentration of the culture medium can be monitored using known techniques, such as, for example, use of the glucose oxidase enzyme test or high pressure liquid chromatography, which can be used to monitor glucose concentration in the supernatant, e.g., a cell-free component of the culture medium. The carbon source concentration is typically maintained below the level at which cell growth inhibition occurs. Although such concentration may vary from organism to organism, for glucose as a carbon source, cell growth inhibition occurs at glucose concentrations greater than at about 60 g/L, and can be determined readily by trial. Accordingly, when glucose is used as a carbon source the glucose is preferably fed to the fermentor and maintained below detection limits. Alternatively, the glucose concentration in the culture medium is maintained in the range of from about 1 g/L to about 100 g/L, more preferably in the range of from about 2 g/L to about 50 g/L, and yet more preferably in the range of from about 5 g/L to about 20 g/L. Although the carbon source concentration can be maintained within desired levels by addition of, for example, a substantially pure glucose solution, it is acceptable, and may be preferred, to maintain the carbon source concentration of the culture medium by addition of aliquots of the original culture medium. The use of aliquots of the original culture medium may be desirable because the concentrations of other nutrients in the medium (e.g. the nitrogen and phosphate sources) can be maintained simultaneously. Likewise, the trace metals concentrations can be maintained in the culture medium by addition of aliquots of the trace metals solution.

Other suitable fermentation medium and methods are described in, e.g., WO 2016/196321.

### 5.8 Fermentation Compositions

In another aspect, provided herein are fermentation compositions comprising a genetically modified host cell described herein and steviol glycosides produced from genetically modified host cell. The fermentation compositions may further comprise a medium. In certain embodiments, the fermentation compositions comprise a genetically modified host cell, and further comprise RebA, RebD, and RebM. In certain embodiments, the fermentation compositions provided herein comprise RebM as a major component of the steviol glycosides produced from the genetically modified host cell. In certain embodiments, the fermentation compositions comprise RebA, RebD, and RebM at a ratio of at least 1:7:50. In certain embodiments, the fermentation compositions comprise RebA, RebD, and RebM at a ratio of at least 1:7:50 to 1:100:1000. In certain embodiments, the fermentation compositions comprise a ratio of at least 1:7:50 to 1:200:2000. In certain embodiments, the ratio of RebA, RebD, and RebM are based on the total content of steviol glycosides that are associated with the genetically modified host cell and the medium. In certain embodiments, the ratio of RebA, RebD, and RebM are based on the total content of steviol glycosides in the medium. In certain embodiments, the ratio of RebA. RebD, and RebM are based on the total content of steviol glycosides that are associated with the genetically modified host cell.

In certain embodiments, the fermentation compositions provided herein contain RebM2 at an undetectable level. In certain embodiments, the fermentation compositions provided herein contain non-naturally occurring steviol glycosides at an undetectable level.

### 5.9 Recovery of Steviol Glycosides

Once the steviol glycoside is produced by the host cell, it may be recovered or isolated for subsequent use using any suitable separation and purification methods known in the art. In some embodiments, a clarified aqueous phase comprising the steviol glycoside is separated from the fermentation by centrifugation. In other embodiments, a clarified aqueous phase comprising the steviol glycoside is separated from the fermentation by adding a demulsifier into the fermentation reaction. Illustrative examples of demulsifiers include flocculants and coagulants.

The steviol glycoside produced in these cells may be present in the culture supernatant and/or associated with the host cells. In embodiments where some of the steviol glycoside is associated with the host cell, the recovery of the steviol glycoside may comprise a method of improving the release of the steviol glycosides from the cells. In some embodiments, this could take the form of washing the cells with hot water or buffer treatment, with or without a surfactant, and with or without added buffers or salts. In some embodiments, the temperature is any temperature deemed suitable for releasing the steviol glycosides. In some embodiments, the temperature is in a range from 40 to 95° C.; or from 60 to 90° C.; or from 75 to 85° C. In some embodiments, the temperature is 40, 45, 50, 55, 65, 70, 75, 80, 85, 90, or 95° C. In some embodiments physical or chemical cell disruption is used to enhance the release of steviol glycosides from the host cell. Alternatively and/or subsequently, the steviol glycoside in the culture medium can be recovered using an isolation unit operations including, but not limited to solvent extraction, membrane clarification, membrane concentration, adsorption, chromatography, evaporation, chemical derivatization, crystallization, and drying.

### 5.10 Methods of Making Genetically Modified Cells

Also provided herein are methods for producing a host cell that is genetically engineered to comprise one or more of the modifications described above, e.g., one or more nucleic heterologous nucleic acids encoding Stevia rebaudiana kaurenoic acid hydroxylase, and/or biosynthetic pathway enzymes, e.g., for a steviol glycoside compound. Expression of a heterologous enzyme in a host cell can be accomplished by introducing into the host cells a nucleic acid comprising a nucleotide sequence encoding the enzyme under the control of regulatory elements that permit expression in the host cell. In some embodiments, the nucleic acid is an extrachromosomal plasmid. In other embodiments, the nucleic acid is a chromosomal integration vector that can integrate the nucleotide sequence into the chromosome of the host cell. In other embodiments, the nucleic acid is a linear piece of double stranded DNA that can integrate via homology the nucleotide sequence into the chromosome of the host cell.

Nucleic acids encoding these proteins can be introduced into the host cell by any method known to one of skill in the art without limitation (see, for example, Hinnen et al. (1978) Proc. Natl. Acad. Sci. USA 75:1292-3; Cregg et al. (1985) Mol. Cell. Biol. 5:3376-3385; Goeddel et al. eds, 1990, Methods in Enzymology, vol. 185, Academic Press, Inc., CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, NY; and Ausubel et al., eds., Current Edition, Current Protocols in Molecular Biology, Greene Publishing Associates and Wiley Interscience, NY). Exemplary techniques include, but are not limited to, spheroplasting, electroporation, PEG 1000 mediated transformation, and lithium acetate or lithium chloride mediated transformation.

The amount of an enzyme in a host cell may be altered by modifying the transcription of the gene that encodes the enzyme. This can be achieved for example by modifying the copy number of the nucleotide sequence encoding the enzyme (e.g., by using a higher or lower copy number expression vector comprising the nucleotide sequence, or by introducing additional copies of the nucleotide sequence into the genome of the host cell or by deleting or disrupting the nucleotide sequence in the genome of the host cell), by changing the order of coding sequences on a polycistronic mRNA of an operon or breaking up an operon into individual genes each with its own control elements, or by increasing the strength of the promoter or operator to which the nucleotide sequence is operably linked. Alternatively or in addition, the copy number of an enzyme in a host cell may be altered by modifying the level of translation of an mRNA that encodes the enzyme. This can be achieved for example by modifying the stability of the mRNA, modifying the sequence of the ribosome binding site, modifying the distance or sequence between the ribosome binding site and the start codon of the enzyme coding sequence, modifying the entire intercistronic region located “upstream of” or adjacent to the 5′ side of the start codon of the enzyme coding region, stabilizing the 3′-end of the mRNA transcript using hairpins and specialized sequences, modifying the codon usage of enzyme, altering expression of rare codon tRNAs used in the biosynthesis of the enzyme, and/or increasing the stability of the enzyme, as, for example, via mutation of its coding sequence.

The activity of an enzyme in a host cell can be altered in a number of ways, including, but not limited to, expressing a modified form of the enzyme that exhibits increased or decreased solubility in the host cell, expressing an altered form of the enzyme that lacks a domain through which the activity of the enzyme is inhibited, expressing a modified form of the enzyme that has a higher or lower Kcat or a lower or higher Km for the substrate, or expressing an altered form of the enzyme that is more or less affected by feed-back or feed-forward regulation by another molecule in the pathway.

In some embodiments, a nucleic acid used to genetically modify a host cell comprises one or more selectable markers useful for the selection of transformed host cells and for placing selective pressure on the host cell to maintain the foreign DNA.

In some embodiments, the selectable marker is an antibiotic resistance marker. Illustrative examples of antibiotic resistance markers include, but are not limited to, the BLA, NAT1, PAT, AUR1-C, PDR4, SMR1, CAT, mouse dhfr, HPH, DSDA, KAN^{R}, and SH BLE gene products. The BLA gene product from E. coli confers resistance to beta-lactam antibiotics (e.g., narrow-spectrum cephalosporins, cephamycins, and carbapenems (ertapenem), cefamandole, and cefoperazone) and to all the anti-gram-negative-bacterium penicillins except temocillin; the NAT1 gene product from S. noursei confers resistance to nourseothricin; the PAT gene product from S. viridochromogenes Tu94 confers resistance to bialophos; the AUR1-C gene product from Saccharomyces cerevisiae confers resistance to Auerobasidin A (AbA); the PDR4 gene product confers resistance to cerulenin; the SMR1 gene product confers resistance to sulfometuron methyl; the CAT gene product from Tn9 transposon confers resistance to chloramphenicol; the mouse dhfr gene product confers resistance to methotrexate; the HPH gene product of Klebsiella pneumonia confers resistance to Hygromycin B; the DSDA gene product of E. coli allows cells to grow on plates with D-serine as the sole nitrogen source; the KAN^{R} gene of the Tn903 transposon confers resistance to G418; and the SH BLE gene product from Streptoalloteichus hindustanus confers resistance to Zeocin (bleomycin). In some embodiments, the antibiotic resistance marker is deleted after the genetically modified host cell disclosed herein is isolated.

In some embodiments, the selectable marker rescues an auxotrophy (e.g., a nutritional auxotrophy) in the genetically modified microorganism. In such embodiments, a parent microorganism comprises a functional disruption in one or more gene products that function in an amino acid or nucleotide biosynthetic pathway and that when non-functional renders a parent cell incapable of growing in media without supplementation with one or more nutrients. Such gene products include, but are not limited to, the HIS3, LEU2, LYS1, LYS2, MET15, TRP1, ADE2, and URA3 gene products in yeast. The auxotrophic phenotype can then be rescued by transforming the parent cell with an expression vector or chromosomal integration construct encoding a functional copy of the disrupted gene product, and the genetically modified host cell generated can be selected for based on the loss of the auxotrophic phenotype of the parent cell. Utilization of the URA3, TRP1, and LYS2 genes as selectable markers has a marked advantage because both positive and negative selections are possible. Positive selection is carried out by auxotrophic complementation of the URA3, TRP 1, and LYS2 mutations, whereas negative selection is based on specific inhibitors, i.e., 5-fluoro-orotic acid (FOA), 5-fluoroanthranilic acid, and aminoadipic acid (aAA), respectively, that prevent growth of the prototrophic strains but allows growth of the URA3, TRP1, and LYS2 mutants, respectively. In other embodiments, the selectable marker rescues other non-lethal deficiencies or phenotypes that can be identified by a known selection method.

Described herein are specific genes and proteins useful in the methods, compositions and organisms of the disclosure; however, it will be recognized that absolute identity to such genes is not necessary. For example, changes in a particular gene or polynucleotide comprising a sequence encoding a polypeptide or enzyme can be performed and screened for activity. Typically, such changes comprise conservative mutations and silent mutations. Such modified or mutated polynucleotides and polypeptides can be screened for expression of a functional enzyme using methods known in the art.

Due to the inherent degeneracy of the genetic code, other polynucleotides which encode substantially the same or functionally equivalent polypeptides can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.” Codon optimization for other host cells can be readily determined using codon usage tables or can be performed using commercially available software, such as CodonOp (www.idtdna.com/CodonOptfrom) from Integrated DNA Technologies.

Optimized coding sequences containing codons preferred by a particular prokaryotic or eukaryotic host (Murray et al., 1989, Nucl Acids Res. 17: 477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24: 216-8).

Those of skill in the art will recognize that, due to the degenerate nature of the genetic code, a variety of DNA molecules differing in their nucleotide sequences can be used to encode a given enzyme of the disclosure. The native DNA sequence encoding the biosynthetic enzymes described above are referenced herein merely to illustrate an embodiment of the disclosure, and the disclosure includes DNA molecules of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for the compositions and methods provided herein are encompassed by the disclosure. In some embodiments, two proteins (or a region of the proteins) are substantially homologous when the amino acid sequences have at least about 30%, 40%, 50%, 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (See, e.g., Pearson W. R., 1994, Methods in Mol Biol 25: 365-89).

The following six groups each contain amino acids that are conservative substitutions for one another: 1) Serine(S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. A typical algorithm used comparing a molecule sequence to a database containing a large number of sequences from different organisms is the computer program BLAST. When searching a database containing sequences from a large number of different organisms, it is typical to compare amino acid sequences.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in yeast.

In addition, genes encoding these enzymes can be identified from other fungal and bacterial species and can be expressed for the modulation of this pathway. A variety of organisms could serve as sources for these enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorpha, Candida spp., Trichosporon spp., Yamadazyma spp., including Y. spp. stipitis, Torulaspora pretoriensis, Issatchenkia orientalis, Schizosaccharomyces spp., including S. pombe. Cryptococcus spp., Aspergillus spp., Neurospora spp., or Ustilago spp. Sources of genes from anaerobic fungi include, but are not limited to, Piromyces spp., Orpinomyces spp., or Neocallimastix spp. Sources of prokaryotic enzymes that are useful include, but are not limited to, Escherichia. coli, Zymomonas mobilis, Staphylococcus aureus, Bacillus spp., Clostridium spp., Corynebacterium spp., Pseudomonas spp., Lactococcus spp., Enterobacter spp., and Salmonella spp.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous UDP glycosyltransferases, KAH, or any biosynthetic pathway genes, proteins, or enzymes, techniques may include, but are not limited to, cloning a gene by PCR using primers based on a published sequence of a gene/enzyme of interest, or by degenerate PCR using degenerate primers designed to amplify a conserved region among a gene of interest. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K., Branched-Chain Amino Acids Methods Enzymology, 1970), then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, or MetaCYC. The candidate gene or enzyme may be identified within the above-mentioned databases in accordance with the teachings herein.

### VI. EXAMPLES

### Example 1: Yeast Culturing Conditions

Each DNA construct was integrated into Saccharomyces cerevisiae (CEN.PK113-7D) using standard molecular biology techniques in an optimized lithium acetate transformation. Briefly, cells were grown overnight in yeast extract peptone dextrose (YPD) media at 30° C. with shaking (200 rpm), diluted to an OD600 of 0.1 in 100 mL YPD, and grown to an OD600 of 0.6-0.8. For each transformation, 5 mL of culture were harvested by centrifugation, washed in 5 mL of sterile water, spun down again, resuspended in 1 mL of 100 mM lithium acetate, and transferred to a microcentrifuge tube. Cells were spun down (13,000×g) for 30 s, the supernatant was removed, and the cells were resuspended in a transformation mix consisting of 240 μL 50% PEG, 36 μL 1 M lithium acetate, 10 μL boiled salmon sperm DNA, and 74 μL of donor DNA. For transformations that require expression of the endonuclease F-Cph1, the donor DNA included a plasmid carrying the F-CphI gene expressed under the yeast TDH3 promoter. F-CphI endonuclease expressed in such a manner cuts a specific recognition site engineered in a host strain to facilitate integration of the target gene of interest. Following a heat shock at 42° C. for 40 min, cells were recovered overnight in YPD media before plating on selective media. DNA integration was confirmed by colony PCR with primers specific to the integrations.

### Example 2: Generation of a Base Strain Capable of High Flux to Farnesylpyrophosphate (FPP) and the Isoprenoid Farnesene

A farnesene production strain was created from a wild type Saccharomyces cerevisiae strain (CEN.PK113-7D) by expressing the genes of the mevalonate pathway under the control of native GAL promoters. This strain comprised the following chromosomally integrated mevalonate pathway genes from S. cerevisiae:acetyl-CoA thiolase. HMG-COA synthase, HMG-CoA reductase, mevalonate kinase, phosphomevalonate kinase, mevalonate pyrophosphate decarboxylase, and IPP:DMAPP isomerase. In addition, the strain contained multiple copies of farnesene synthase from Artemisia annua, also under the control of either native GAL1 or GAL10 promoters. All heterologous genes described herein were codon optimized using publicly available or other suitable algorithms. The strain also contained a deletion of the GAL80 gene, and the ERG9 gene encoding squalene synthase is downregulated by replacing the native promoter with the promoter of the yeast gene MET3. Examples of methods of creating S. cerevisiae strains with high flux to isoprenoids are described in the U.S. Pat. Nos. 8,415,136 and 8,236,512 which are incorporated herein in their entireties.

### Example 3: Construction of a Series of Strains for Rapid Screening for Novel Kaurenoic Acid Hydroxylase P450 Enzymes

FIG. 1 shows an exemplary biosynthetic pathway from FPP to Reb M with the kaurenoic acid intermediate. The farnesene base strain described above was further engineered to have high flux to the C20 isoprenoid kaurene by integrating six copies of a geranyl¬geranyl¬pyro¬phosphate synthase (GGPPS) into the genome, one copy of a copalyl¬diphosphate synthase, and four copies of a kaurene synthase. Subsequently, all copies of farnesene synthase were removed from the strain and the strain was confirmed to produce ent-kaurene and no farnesene.

Kaurenoic acid hydroxylase (KAH) is a cytochrome P450 enzyme that catalyzes the oxidation of kaurenoic acid to produce steviol (see FIG. 1) which is necessary to produce Reb M. To screen novel P450 enzymes for KAH activity in vivo in S. cerevisiae, several strains were made that contained all the genes necessary to produce Reb M, except they lacked any copy of a KAH gene. Table 1 lists all Reb M pathway genes and promoters used. The strains containing all genes described in Table 1 primarily produce kaurenoic acid, the substrate for KAH.

TABLE 1
Genes, promoters, and amino acid sequences of the enzymes
used to convert FPP to Reb M.

| Enzyme | SEQ ID | Promoter |
|---|---|---|
| Bt.GGPPS | SEQ ID NO: 7 | PGAL1 |
| Ent-Os.CDPS | SEQ ID NO: 8* | PGAL1 |
| Ent-Pg.KS | SEQ ID NO: 9 | PGAL1 |
| Sr.KO | SEQ ID NO: 6 | PGAL1 |
| At.CPR | SEQ ID NO: 11 | PGAL3 |
| UGT85C2 | SEQ ID NO: 12 | PGAL10 |
| UGT74G1 | SEQ ID NO: 13 | PGAL1 |
| UGT91D_like3 | SEQ ID NO: 14 | PGAL1 |
| UGT76G1 | SEQ ID NO: 15 | PGAL10 |
| UGT40087 | SEQ ID NO: 16 | PGAL1 |

*First 65 amino acids replaced with a single methionine.

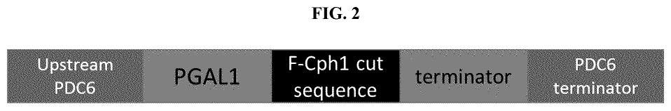

To measure the activity of KAH variants in vivo in S. cerevisiae, initially a first screening strain was constructed that contains all the genes necessary to produce the monoglycosylated steviol metabolite 19-glycoside (Table 1 and FIG. 1), except that it lacks any copy of a KAH gene. Instead, it contains a landing pad to allow for the rapid insertion of KAH variants (FIG. 2). The landing pad consists of 500 bp of locus-targeting DNA sequences on either end of the construct to the genomic region upstream and downstream of the yeast locus of choice (Upstream locus and Downstream locus), thereby deleting the locus when the landing pad is integrated into the yeast chromosome. Internally, the landing pad contains a promoter (Promoter) which can be GAL1, GAL3 or any other promoter of yeast GAL regulon and a yeast terminator of choice (Terminator) flanking an endonuclease recognition site (F-CphI). DNA variants of Sr.KAH (SEQ ID NO: 1) were used to transform the strain along with a plasmid expressing endonuclease F-CphI, which cuts the recognition sequence, creating a double strand break at the landing pad, and facilitating homologous recombination of the Sr.KAH DNA variants at the site.

A second screening strain was derived from the first screening strain that lacks a functional KAH gene by introducing additional genes to ultimately contain all the genes necessary to produce Reb M (Table 1 and FIG. 1). As with the first screening strain, the second screening strain lacks any copy of a KAH gene and contains a cleavable landing pad (FIG. 2) instead.

A third screening strain was generated that has the same engineering as the second screening strain except the Sr.KO was replaced with Ps.KO (SEQ ID NO: 10). The Ps.KO enzyme is described in PCT/US2018/046359 (PISUM SATIVUM KAURENE OXIDASE FOR HIGH EFFICIENCY PRODUCTION OF REBAUDIOSIDES filed Aug. 10, 2018) and is significantly more active in converting kaurene to kaurenoic acid (FIG. 1). The third screening strain therefore has a higher carbon flux to kaurenoic acid, the substrate of the KAH P450. The second and third screening strains are referred to as Reb M producing yeast that lack a functional KAH gene.

### Example 4: Yeast Culturing Conditions

Yeast colonies verified to contain the expected KAH gene were picked into 96-well microtiter plates containing Bird Seed Media (BSM, originally described by van Hoek et al., Biotechnology and Bioengineering 68(5), 2000, pp. 517-523) with 20 g/L sucrose, 37.5 g/L ammonium sulfate, and from 1 to 5 g/L lysine. Cells were cultured at 30° C. in a high capacity microtiter plate incubator shaking at 1000 rpm and 80% humidity for 3 days until the cultures reached carbon exhaustion. The growth-saturated cultures were subcultured into fresh plates containing BSM with 40 g/L sucrose, 150 g/L ammonium sulfate, and 1 g/L lysine by taking 14.4 μL from the saturated cultures and diluting into 360 μL of fresh media. Cells in the production media were cultured at 30° C. in a high capacity microtiter plate shaker at 1000 rpm and 80% humidity for additional 3 days prior to extraction and analysis.

### Example 5: Yeast Sample Preparation Conditions for Analysis of Pathway Intermediates from Farnesol to Rebaudioside M

To extract all steviol glycosides made by cells (see FIG. 1), upon culturing completion, the whole cell broth was diluted with 628 μL of 100% ethanol, sealed with a foil seal, and shaken at 1250 rpm for 30 s. 314 μL of water was added to each well directly to dilute the extraction. The plate was briefly centrifuged to pellet solids. 198 μL of 50:50 ethanol:water containing 0.48 mg/L rebaudioside N (used as an internal standard) was transferred to a new 250 μL assay plate and 2 μL of the culture/ethanol mixture was added to the assay plate. A foil seal was applied to the plate for analysis.

To extract pathway intermediates from farnesol to steviol (see FIG. 1) made by cells, upon culturing completion, the whole cell broth was diluted with 100 μL of 100% methanol, and 500 μL of 100% ethyl acetate, sealed with a foil seal, and shaken at 1000 rpm for 30 min to extract the analytes of interest into the ethyl acetate. The mixture was centrifuged to pellet any solids. 150 μL of the ethyl acetate layer is added to a new 1.1 mL assay plate and 150 μL of fresh ethyl acetate layer added to the assay plate followed by GC-FID analysis.

### Example 6: Analytical Methods

Samples for steviol glycosides measurements were analyzed by mass spectrometer (Agilent 6470-QQQ) with a RapidFire 365 system autosampler with C8 cartridge.

TABLE 2
RapidFire 365 system configuration.

| Pump 1, Line A: 2 mM | 100% A, 1.5 mL/min | |
|---|---|---|
| ammonium formate in water | | |
| Pump 2, Line A: 35% | 100% A, 1.5 mL/min | |
| acetonitrile in water | | |
| Pump 3, Line A: 80% | 100% A, 0.8 mL/min | |
| acetonitrile in water | | |
| State 1: Aspirate | 600 | ms |
| State 2: Load/Wash | 3000 | ms |
| State 3: Extra wash | 1500 | ms |
| State 4: Elute | 5000 | ms |
| State 5: Reequilibrate | 1000 | ms |

TABLE 3
6470-QQQ MS method configurations.

| Ion Source | AJS ESI | |
|---|---|---|
| Time Filtering peak width | 0.02 | min |
| Stop Time | No limit/as pump | |
| Scan Type | MRM | |
| Diverter Valve | To MS | |
| Delta EMV | (+)0/(−)300 | |
| Ion Mode (polarity) | Negative | |
| Gas Temp | 250° | C. |
| Gas Flow | 11 | L/min |
| Nebulizer | 30 | psi |
| Sheath Gas Temp | 350° | C. |
| Sheath Gas Flow | 11 | L/min |
| Negative Capillary V | 2500 | V |

The peak areas from a chromatogram from a mass spectrometer were used to generate the calibration curve using authentic standards. The molar ratios of relevant compounds were determined by quantifying the amount in moles of each compound through external calibration using an authentic standard, and then taking the appropriate ratios.

Samples upstream of steviol glycosides (i.e. farnesol through steviol, FIG. 1) were analyzed by gas chromatography with flame ionization detection (GC-FID, Agilent 7890A) using an Agilent DB-17 MS (20 m×0.18 mm×0.18 μm, P/N 121-4722) in split mode with a split ratio of 50. The following temperature and flow gradients were used:

TABLE 4
Temperature gradient.

| | |
|---|---|
| Initial Temp, (° C.) | 140 |
| Initial Hold, (min) | 0.0 |
| Rate 1, (° C./min) | 15 |
| Temp 1, (° C.) | 175 |
| Hold Time 1, (min) | 0.0 |
| Rate 2, (° C./min) | 100 |
| Temp 2, (° C.) | 320 |
| Hold Time 2, (min) | 2.0 |
| Runtime (min) | 6.21 |

TABLE 5
Flow gradient.

| Carrier gas | Hydrogen |
|---|---|
| Pressure, psi | 47.2 |
| Flow, mL/min at t = 0 min | 3.0 |
| Velocity, cm/s | 85.05 |
| Mode | Ramped flow |
| Flow 1, (mL/min) | 3.0 |
| Hold Time 1, (min) | 5.5 |
| Rate 2, (mL/min/min) | 10.0 |
| Flow 2 (mL/min) | 5.0 |
| Hold Time 2, (min) | 0.0 |

Each analyte was identified by retention time, determined from an authentic standard. The peak areas from a chromatogram are used to generate the calibration curve. The molar ratio of kaurene, kaurenol, kaurenal, kaurenoic acid, and steviol were determined by quantifying the amount in moles of each compound through external calibration using an authentic standard, and then taking the appropriate ratios.

### Example 7: Evolution of Stevia rebaudiana Kaurenoic Acid Hydroxylase Via Site Directed Saturation Mutagenesis

In this example, activity data is provided for a P450 enzyme (kaurenoic acid hydroxylase from the plant Stevia rebaudiana, Sr.KAH) and specific mutations that improve Sr.KAH activity expressed in S. cerevisiae in vivo to produce steviol glycosides and Reb M.

KAH is a cytochrome P450 enzyme that catalyzes the oxidation of kaurenoic acid to steviol (FIG. 1) which is necessary for the formation of Reb M. Each amino acid residue in Sr.KAH was mutated to 12 different amino acids (R, N, D, C, G, H, I, L, F, S, Y, and V) that are coded by the degenerate codon NDT where N stands for any (nucleotide adenine, thymine, guanine and cytosine); D stands for adenine, guanine and thymine; and T stands for thymine. The NDT library of Sr.KAH gene variants was constructed via PCR using primers containing an NDT degenerate codon at the desired position. Each PCR product contains a mixture of gene variants so that 12 possible different amino acids are encoded at a specific position corresponding to a single protein residue. In each PCR product, the pool of Sr.KAH gene variants are flanked on both ends by 40 bp of sequences homologous to the promoter (at 5′ of the gene) and the terminator (at 3′ of the gene) regions of the landing pad incorporated at the specific locus of S. cerevisiae host strain (see FIG. 2).

To measure the activity of KAH variants generated in the first round of site saturation mutagenesis in S. cerevisiae in vivo, the mutated Sr.KAH variants were used to transform either a 19-glycoside producing yeast or a Reb M producing yeast that lacked functional KAH genes. Wild type Sr.KAH was used as a control. The in vivo KAH activity was measured in an initial Tier 1 screen as the titer of either 19-glycoside or Reb M, respectively. In the Tier 1 screen, twenty-four isolates were screened per mutated codon position giving a 2× coverage at N=1.

For screening in the 19-glycoside producing yeast, the effect of each mutation was calculated by normalizing the amount of 19-glycoside produced in a strain containing an Sr.KAH mutant allele to the amount of 19-glycoside produced by a strain containing the wild type Sr.KAH allele. For screening in Reb M producing yeast, all the steviol glycosides produced by a strain were extracted and measured via mass spectrometry. The sum of all the steviol glycosides was calculated (in μM); this measurement is called the “steviol equivalent.” The effect of a mutation was calculated by normalizing the steviol equivalent of a strain containing a Sr.KAH mutant allele to the steviol equivalent of a strain containing the wild type Sr.KAH allele.

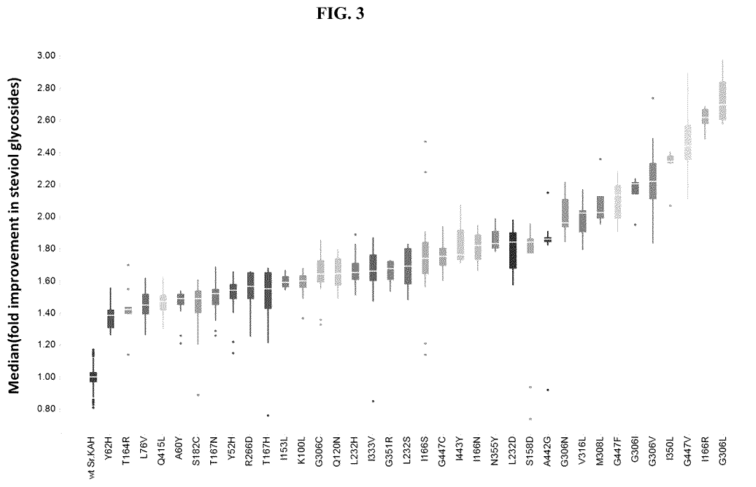

Upon finding mutations in Tier 1 that appear to increase activity of the Sr.KAH enzyme in vivo, a Tier 2 screen was performed with higher replication (N=12) of strains containing a specific mutant of interest to confirm the improvement, using the same calculations as above. The resulting Sr.KAH mutants, each containing a single amino acid change, that have activity at least one standard deviation higher than the wild type Sr.KAH allele is reported in FIG. 3 and Table 6 as the fold increase in steviol equivalents over the wild type parent Sr.KAH allele. For example, if a mutant strain has 2-fold (2×) higher activity, then it produced double the amount (or 100% more) of measured steviol glycosides (steviol equivalents) over a strain with wild type Sr.KAH. The best mutant from the site directed saturation mutagenesis NDT library (G306L) provides a 2.7-fold improvement of in vivo KAH activity compared to wild type Sr.KAH.

TABLE 6
Improved alleles of Sr.KAH: the associated amino acid changes
and fold improvement over wild type Sr.KAH activity.

| | Fold | Percent |
|---|---|---|
| Sr.KAH sequence variations | improvement | improvement |
| compared to wild type | over wt Sr.KAH | over wt Sr.KAH |
| wild type Sr.KAH | 1.00 | 0 |
| Y62H | 1.39 | 39 |
| T164R | 1.43 | 43 |
| L76V | 1.45 | 45 |
| Q415L | 1.47 | 47 |
| A60Y | 1.49 | 49 |
| S182C | 1.52 | 52 |
| T167N | 1.52 | 52 |
| Y52H | 1.54 | 54 |
| R266D | 1.57 | 57 |
| T167H | 1.58 | 58 |
| I153L | 1.59 | 59 |
| K100L | 1.60 | 60 |
| G306C | 1.64 | 64 |
| Q120N | 1.65 | 65 |
| L232H | 1.65 | 65 |
| I333V | 1.66 | 66 |
| G351R | 1.68 | 68 |
| L232S | 1.69 | 69 |
| I166S | 1.74 | 74 |
| W447C | 1.75 | 75 |
| I443Y | 1.76 | 76 |
| I166N | 1.82 | 82 |
| N355Y | 1.83 | 83 |
| L232D | 1.84 | 84 |
| S158D | 1.86 | 86 |
| A442G | 1.86 | 86 |
| G306N | 1.96 | 96 |
| V316L | 2.02 | 102 |
| M308L | 2.03 | 103 |
| G447F | 2.14 | 114 |
| G306I | 2.21 | 121 |
| G306V | 2.22 | 122 |
| I350L | 2.35 | 135 |
| G447V | 2.44 | 144 |
| I166R | 2.62 | 162 |
| G306L | 2.70 | 170 |

### Example 8: Combinatorial Library of Single Mutations to Improve Sr.KAH Activity

Two sets of 12 mutations were selected from the unique NDT library hits to build full factorial combination libraries. Each set contained nine of the same mutations and three unique mutations per set. The first combinatorial library contained mutations K100L, Q120N, I153L, S158D, I166R, L232D, G306L, V316L, I333V, I350L, A442G, G447V, and the second library contained mutations S158D, I166R, L232D, G306L, M308L, V316L, I333V, I350L, N355Y, A442G, I443Y, G447V. The libraries were designed to create all possible combinations among the 12 mutations to find the combination that leads to the highest activity of Sr.KAH in vivo. The genes were assembled from a mixture of custom built gBlocks (Integrated DNA Technologies, Inc.) with overlapping homology on the ends of each piece so that the pieces overlapped in sequence; assembling all the pieces together reconstituted a full length KAH allele. The terminal 5′ and 3′ pieces also had homology to the promoter and terminator of the landing pad sequence in Reb M producing yeast that lack a functional KAH gene. The pieces were transformed into yeast and relied on endogenous homologous recombination to assemble the pieces together.

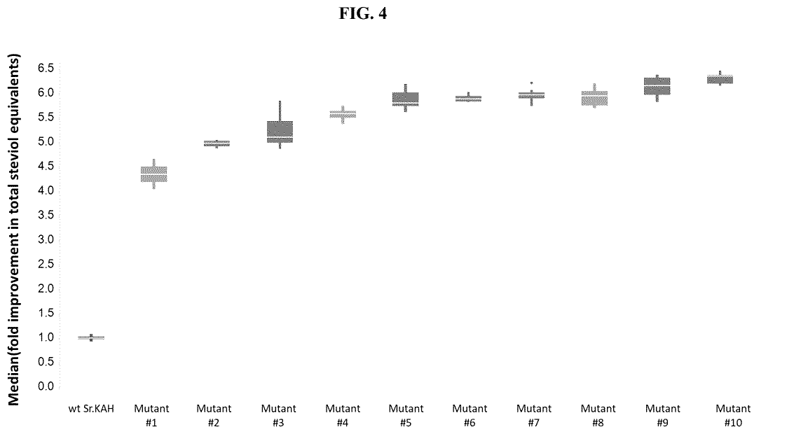

The Tier 1 combinatorial library DNA was screened in the Reb M producing yeast at a 2× coverage. The top 10 KAH alleles from the combination library screen were PCR-amplified and the DNA was used to transform a Reb M producing yeast as a Tier 2 confirmation at N=4 replication. The simplest, improved KAH combinatorial variant contained two mutations at I166R and I333V and was 4.3-fold improved in comparison to wild type Sr.KAH. The top combinatorial KAH mutants improved total steviol production 5-fold and 6.3-fold over wild type Sr.KAH (FIG. 4 and Table 7). The best combination of amino acid substitutions (6.3× improvement over wild type Sr.KAH in the Reb M producing yeast) comprised the mutations S158D, I166R, G306L, M308L, V316L, and I350L.

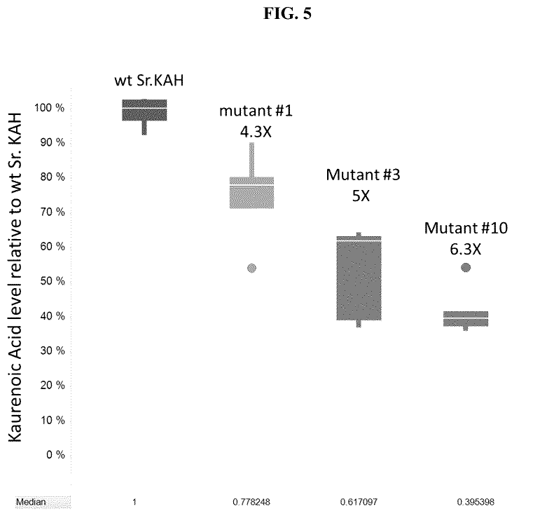

Improved KAH mutants show a reduction in the substrate kaurenoic acid compared to wild type Sr.KAH (FIG. 5), again demonstrating that Sr.KAH alleles with improved activity are converting more substrate to steviol, thereby increasing Reb M production in yeast in vivo. In FIG. 5, the amount of kaurenoic acid is normalized to that in the strain containing wild type Sr.KAH (100%). The reduction in kaurenoic acid levels in the yeast containing improved Sr.KAH alleles is shown as a percent of the kaurenoic acid levels in wild type Sr.KAH. The Sr.KAH allele with the most improved activity has kaurenoic acid levels that are 40% of the amount of kaurenoic acid that is observed in strains with wild type Sr.KAH.

TABLE 7
Improved alleles of Sr.KAH, fold improvement over wild type
Sr.KAH activity, and the associated amino acid changes.

| | Fold | Percent | | | | | | |
|---|---|---|---|---|---|---|---|---|
| | improvement | improvement | | | | | | |
| Sr.KAH | over wt | over wt | List of sequence variations | | | | | |
| alleles | Sr.KAH | Sr.KAH | compared to wild type | | | | | |
| wild type | 1.00 | 0 | | | | | | |
| Sr.KAH | | | | | | | | |
| mutant #1 | 4.34 | 334 | I166R | I333V | | | | |
| mutant #2 | 4.98 | 398 | I166R | I350L | G447V | | | |
| mutant #3 | 5.11 | 411 | I153L | S158D | I166R | L232D | I333V | I350L |
| mutant #4 | 5.58 | 458 | S158D | G306L | I350L | | | |
| mutant #5 | 5.8 | 480 | G306L | V316L | I350L | G447V | | |
| mutant #6 | 5.88 | 488 | G306L | V316L | I333V | I350L | | |
| mutant #7 | 5.95 | 495 | S158D | I166R | L232D | G306L | I333V | |
| mutant #8 | 5.96 | 496 | L232D | G306L | V316L | I333V | I350L | |
| mutant #9 | 6.16 | 516 | I166R | L232D | G306L | I350L | | |
| mutant #10 | 6.36 | 536 | S158D | I166R | G306L | M308L | V316L | I350L |

### Example 9: N-Terminal Domain Swaps to Improve in Vivo Activity of Wild Type Sr.KAH

This example provides modified kaurenoic acid hydroxylase polypeptides with substituted N-terminal domains that show improved activity.

Kaurenoic acid hydroxy lase is a cytochrome P450 enzyme. Most eukaryotic P450s are membrane-bound proteins, and the high-level domain structure of membrane-associated cytochrome P450 enzymes is highly conserved. Plant cytochrome P450 enzymes are incorporated into the endoplasmic reticulum (ER) with a long N-terminal polypeptide chain of roughly 30-50 amino acids that mediates membrane targeting. The catalytic domain of the P450 enzymes face the cytoplasmic side of the endoplasmic reticulum. The region of the N-terminus that is inserted into the ER membrane stops at the end of a hydrophobic stretch of roughly 20 amino acid residues and precedes the catalytic domain. A short region that generally contains positively charged residues links the catalytic domain to a conserved proline rich motif in the N-terminus of the structurally conserved P450 fold. The N-terminal ER-targeting domain is unlikely to be closely associated with the catalytic domain, and the N-terminal domain is not required for function. However, interactions between the P450 catalytic domain and the ER membrane are important for protein activity. For example, interactions with the ER membrane could promote the transfer of hydrophobic substrates from the membrane to the catalytic site and could also orient the protein to facilitate interaction with a cytochrome P450 reductase (CPR), which is necessary for activity.

While not intending to be bound by any theory of operation, we discovered that swapping the ER-associated N-terminus of a P450 protein with other N-terminal transmembrane domains could lead to an improvement in heterologously expressed plant P450 activity in S. cerevisiae. Swapping N-terminal transmembrane domains should not disturb the ER-targeting, nor should it change the specificity of the catalytic domain, but it might help with ER localization of the heterologous protein and/or it might alter the protein conformation to improve the interactions of the catalytic domain with the ER membrane. This latter effect could lead to improved interactions with ER-bound substrates or the ER-bound CPRs.

This example provides activity data for the wild type kaurenoic acid hydroxylase from the plant Stevia rebaudiana (Sr.KAH) in which the native N-terminal transmembrane domain was swapped with the N-terminal transmembrane domain of other ER-bound proteins, including, but not limited to, other cytochrome P450 enzymes and cytochrome P450 reductases (CPRs).

To make the chimeric proteins, wild type Sr.KAH and the candidate ER-bound proteins were truncated at various positions, such as (1) the proline rich region, (2) the transmembrane domain site predicted from a TMHMM server (available at www.cbs.dtu.dk/services/TMHMM/), (3) the positively charged residues embedded approximately around amino acid number 30-50 of the N-terminus, or (4) based on sequence alignment. The N-terminal region from the candidate proteins was then added to the truncated Sr.KAH protein containing the catalytic domain.

To screen for activity, the genes of the chimeric proteins were used to transform a Reb M producing yeast that lack a functional KAH gene described above; wild type Sr.KAH (SEQ ID NO:1, which corresponds to sequence identification no. 164 in EP 3009508) was used as the control.

Two separate methods were used to calculate KAH P450 function. The first was to take the ratio of total Reb M made in the cell with a chimera protein over the total Reb M made in the cell with wild type Sr.KAH. The second method to calculate P450 function was to sum in μM all the steviol glycosides made in the cell with a chimera protein over the total steviol glycosides in μM made in the cell with wild type Sr.KAH. This latter measure of “steviol equivalents” is more accurate, since it calculates all the steviol made in the cell, even if some intermediates remain and do not get converted all the way to Reb M.

Each chimera was ranked by its total steviol equivalents ratio to the wild type Sr.KAH. The N-terminal domains from plant CPR ATR2 and plant Stevia rebaudiana kaurene oxidase (Sr.KO) showed equal or improved activity relative to wild type Sr.KAH (Table 8).

TABLE 8
Improved alleles of wild type Sr.KAH derivatives resulting
from N-terminal domain swaps, fold improvement over wild type
Sr.KAH activity, and the associated amino acid changes.

| | Length of | | | Reb M | Total steviol |
|---|---|---|---|---|---|
| | N-terminal | Length of | | ratio | glycosides |
| | deletion | new N- | Source of | chimera/ | ratio |
| | (aa) of | terminus | new N- | wt | chimera/wt |
| Polypeptide | Sr.KAH | added | terminus | Sr.KAH | Sr.KAH |
| wild type Sr.KAH | 0 | 0 | NA | 1.00 | 1.00 |
| (SEQ ID NO: 1; | | | | | |
| control) | | | | | |
| ATR2(1a:72a):Sr.KAH(23a:500a) | 22 | 72 | ATR2 | 1.28 | 1.35 |
| SEQ ID NO: 2 | | | | | |
| ATR2(1a:50a):Sr.KAH(5a:500a) | 4 | 50 | ATR2 | 1.16 | 1.29 |
| SEQ ID NO: 3 | | | | | |
| Sr.KO(1a:23a):Sr.KAH(5a:500a) | 4 | 23 | Sr.KO | 1.16 | 1.01 |
| SEQ ID NO: 5 | | | | | |

### Example 10: N-Terminal Domain Swaps to Improve in Vivo Activity of the Best Evolved Variant of Sr.KAH, Mutant #10

The amino acid mutations found in most improved combinatorial Sr.KAH variant in Example 8, mutant #10, were combined with the three N-terminal domain swaps in Example 9, Table 8 that increased total steviol equivalents. Chimeras were made exactly as described in Example 9, only instead of using wild type Sr.KAH DNA sequence, the DNA sequence of mutant #10 from Example 8 was used. All the mutations in Sr.KAH mutant #10 were retained in the resulting N-terminal domain-swapped chimeras. The new chimeric genes, containing the evolved amino acid mutations, were transformed into Reb M producing yeast. The evolved Sr.KAH mutant #10 was used as the experimental control. The effect of the N-terminal domain swaps on activity of Sr.KAH mutant #10 was calculated exactly as was described in Example 9. Fusion of N-terminus sequence from two genes, the CPRs from Artemisia annua (Aa.CPR) and Arabidopsis thaliana (ATR2), to the N-terminally truncated Sr.KAH mutant #10 resulted in improvements as high as 60% in KAH enzymatic activity (Table 9).

TABLE 9
Alleles of evolved Sr.KAH mutant #10 resulting from N-terminal domain swaps, fold
improvement over Sr.KAH mutant #10 activity, and the associated amino acid changes.

| | Length of | | | | Total steviol |
|---|---|---|---|---|---|
| | N-terminal | | | Reb M | glycosides |
| | deletion | Length of | | ratio | ratio |
| | (aa) of | new N- | Source of | chimera/ | chimera/ |
| | Sr.KAH | terminus | new N- | Sr.KAH | Sr.KAH |
| Polypeptide | mutant #10 | added | terminus | mutant #10 | mutant #10 |
| Sr.KAH mutant #10 | 0 | 0 | NA | 1.00 | 1.00 |
| (control) | | | | | |
| Aa.CPR(1a:66a):Sr.KAH_mutant#10(23a:500a) | 22 | 66 | Aa.CPR | 1.53 | 1.58 |
| SEQ ID NO: 21 | | | | | |
| ATR2(1a:72a):Sr.KAH_mutant#10(23a:500a) | 22 | 72 | ATR2 | 1.60 | 1.63 |
| SEQ ID NO: 2 | | | | | |

### Example 11: N-Terminal Domain Swaps to Improve in Vivo Activity of Evolved Variant of Sr.KAH, Mutant #3

To test whether beneficial N-terminal domain swaps identified in Examples 9 and 10 can be utilized to improve the activity of other Sr.KAH variants, beneficial amino acid mutations from another improved variant, Sr.KAH mutant #3 (Example 8), were combined with two best N-terminal domain swaps identified in Example 10. The resulting chimeric genes were used to transform Reb M producing yeast that lack a functional KAH gene and titers of Reb M and total steviol glycosides were analyzed (Table 10). Fusion of N-terminus sequence from two genes, the CPRs from Artemisia annua (Aa.CPR) and Arabidopsis thaliana (ATR2), to the 22 amino acid N-terminally truncated Sr.KAH mutant #3 resulted in improvements of approximately 25% in this strain background.

TABLE 10
Alleles of evolved Sr.KAH mutant #3 resulting from N-terminal domain swaps,
fold improvement over Sr.KAH mutant #3 activity, and the associated amino acid changes.

| | Length of | | | | Total steviol |
|---|---|---|---|---|---|
| | N-terminal | | | Reb M | glycosides |
| | deletion | Length of | | ratio | ratio |
| | (aa) of | new N- | Source of | chimera/ | chimera/ |
| | Sr.KAH | terminus | new N- | Sr.KAH | Sr.KAH |
| Polypeptide | mutant #3 | added | terminus | mutant #3 | mutant #3 |
| Sr.KAH mutant #3 | 0 | 0 | NA | 1.00 | 1.00 |
| (control) | | | | | |
| Aa.CPR(1a:66a):Sr.KAH_mutant#3(23a:500a) | 22 | 66 | Aa.CPR | 1.24 | 1.26 |
| SEQ ID NO: 24 | | | | | |
| ATR2(1a:72a):Sr.KAH_mutant#3(23a:500a) | 22 | 72 | ATR2 | 1.27 | 1.28 |
| SEQ ID NO: 25 | | | | | |

### Example 12: N-Terminal Domain Swaps with Native Yeast ER Proteins to Improve in Vivo Activity of Evolved Variant of Sr.KAH, Mutant #10

One of the topology characters shared by the two beneficial transmembrane domains derived from Aa.CPR and ATR2 in Examples 10 and 11 is an approximately 50 amino acid long sequence predicted to be inside the endoplasmic reticulum (ER) lumen preceding the first transmembrane helix. There are 15 proteins in S. cerevisiae documented in the Membranome data base (available at membranome.org/) having a single-helix transmembrane domain at their N-terminus. Scanning these 15 protein sequences using the TMHMM server (available at www.cbs.dtu.dk/services/TMHMM/) revealed several such N-terminal sequences resembling the pattern shared by transmembrane domains from Aa.CPR and ATR2. These include ERG11 (1a:80a), ERG11 (1a:51a), ALG11 (1a:45a), SEC66 (1a:50a), NUS1 (1a:119a), RCR1 (1a:62a), and UBP1 (1a:52a).

The fusions of these N-terminal, native yeast sequences N-terminally truncated Sr.KAH mutant #10 were created. The chimeric gene sequences were inserted into Reb M producing yeast that lack a functional KAH gene and titers of Reb M and total steviol glycosides were analyzed as described in Example 8.

Among the seven chimeric genes tested, two chimeras containing SEC66 (1a:50a) and UBP1 (1a:52a) were improved in comparison to Sr.KAH mutant #10 by approximately 20%. Three N-terminal sequences, ERG11 (1a:80a), NUS1 (1a:119a), and RCR1 (1a:62a), apparently disrupted the function of KAH, whereas two, ALG11 (1a:45a) and ERG11 (1a:51a), had no significant effect on activity (Table 11).

TABLE 11
Alleles of evolved Sr.KAH mutant #10 resulting from native yeast N-terminal domain swaps,
fold improvement over Sr.KAH mutant #10 activity, and the associated amino acid changes.

| | Length N- | | | | Total steviol |
|---|---|---|---|---|---|
| | terminal | | | Reb M | glycosides |
| | deletion | Length of | | ratio | ratio |
| | (aa) of | new N- | Source of | chimera/ | chimera/ |
| | Sr.KAH | terminus | new N- | Sr.KAH | Sr.KAH |
| Polypeptide | mutant #10 | added | terminus | mutant #10 | mutant #10 |
| Sr.KAH mutant #10 | 0 | 0 | NA | 1.00 | 1.00 |
| (control) | | | | | |
| ALG11(1a:45a):Sr.KAH_mutant#10(23a:1503a) | 22 | 45 | ALG11 | 1.06 | 1.08 |
| SEQ ID NO: 26 | | | | | |
| ERG11(1a:51a):Sr.KAH_mutant#10(23a:500a) | 22 | 51 | ERG11 | 1.05 | 1.07 |
| SEQ ID NO: 27 | | | | | |
| ERG11(1a:80a):Sr.KAH_mutant#10(47a:500a) | 46 | 80 | ERG11 | 0.25 | 0.23 |
| SEQ ID NO: 28 | | | | | |
| NUS1(1a:119a):Sr.KAH_mutant#10(23a:500a) | 22 | 119 | NUS1 | 0.41 | 0.38 |
| SEQ ID NO: 29 | | | | | |
| RCR1(1a:62a):Sr.KAH_mutant#10(23a:500a) | 22 | 62 | RCR1 | 0.04 | 0.04 |
| SEQ ID NO: 30 | | | | | |
| SEC66(1a:50a):Sr.KAH_mutant#10(23a:500a) | 22 | 50 | SEC66 | 1.16 | 1.19 |
| SEQ ID NO: 31 | | | | | |
| UBP1(1a:52a):Sr.KAH_mutant#10(23a:500a) | 22 | 52 | UBP1 | 1.18 | 1.20 |
| SEQ ID NO: 32 | | | | | |

Importantly, the results of Examples 9, 10, 11, and 12 demonstrate that swapping the N-terminal, ER-associated protein domain of cytochrome P450 enzymes improves cytochrome P450 enzyme activity when expressing the P450 enzyme in a heterologous host.

### Example 13: Further Improvement of Sr.KAH Mutant #3 Via Site Directed Saturation Mutagenesis

To further improve the activity of Sr.KAH mutant #3 (Example 8) another round of site directed saturation mutagenesis was applied and mutant variants with even higher activity in converting kaurenoic acid to steviol (FIG. 1) were isolated. Each of 175 selected positions (E2, A3, S4, Y5, L6, Y7, I8, I10, L11, L12, L13, L14, A15, S16, Y17, L18, F19, T20, T21, Q22, L23, R24, R25, K26, S27, A28, N29, L30, F35, S37, I38, I40, I41, H43, L46, L47, K49, Y52, T54, K57, I58, A60, Y62, L66, Q67, L68, L70, G71, Y72, R74, L76, S80, P81, S82, E85, C87, T89, N91, V93, I94, F95, N97, K100, L102, K105, I106, V107, T110, S114, S116, D119, Q120, N123, V127, S129, I130, I132, V135, H136, N139, D143, R145, N149, R150, L151, L153, R157, D158, S160, S161, T164, L165, R166, T167, V168, L172, L174, I177, S182, D191, R192, S215, G218, I223, L227, V229, K230, D232, K235, I237, G249, R258, G259, A260, K261, V262, G263, K264, G265, R266, G295, G306, M308, V316, V333, N336, I339, I344, G345, L350, G351, N355, S373, A374, S379, N382, I383, G386, L389, V391, H397, T408, Q415, G416, L417, G419, T420, R421, D422, G423, F424, K425, L426, M427, S431, G432, R433, G440, A442, I443, L445, G447, M448, V453, V462, L473, V484, P489, S491, E492, T494, N495, L496, L497, and S498) in the Sr.KAH mutant #3 sequence was mutated to 15 different amino acids (A, C, D, F, G, H, I, L, N, P, R, S—encoded by two codons—, T, V, and Y) that are encoded by the degenerate codon NNT where N stands for any nucleotide adenine, thymine, guanine and cytosine and T stands for thymine. The NNT library of Sr.KAH gene variants was constructed via PCR using primers containing an NNT degenerate codon at the desired position as described in Example 7 and used to transform Reb M producing yeast that lack a functional KAH gene for integration at the landing pad for expression of KAH.

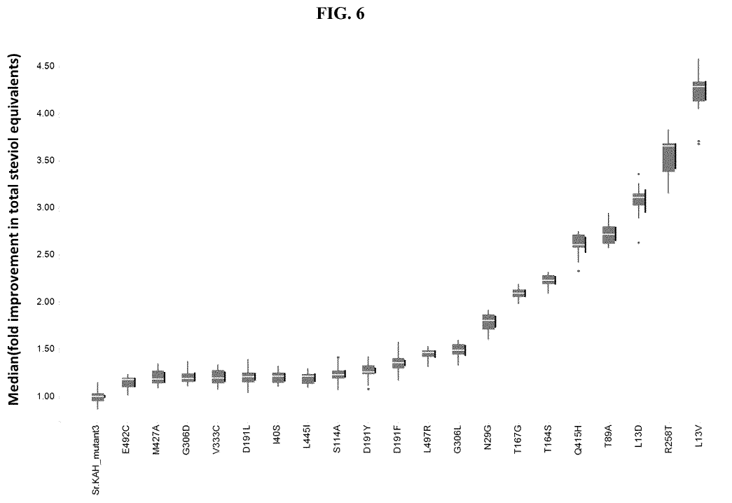

The in vivo KAH activity of the NNT library mutants was measured in a Tier 1 screen versus the Sr.KAH mutant #3 allele using the titer of total steviol glycosides (μM) as described in Example 7. Thirty-nine isolates were screened per mutated position in the protein sequence giving approximately a 2.5× coverage at N=1 for each unique variant. Upon finding mutations in Sr.KAH mutant #3 that appear to increase activity of the enzyme in vivo, a Tier 2 screen was performed with higher replication (N=10) of strains containing a specific mutant of interest to confirm the improvement, using the same calculations as described in Example 7. The resulting activity of an NNT-derived mutant Sr.KAH allele is reported in fold increase over Sr.KAH mutant #3 in FIG. 6 and Table 12.

TABLE 12
Improved alleles of Sr.KAH mutant #3: the associated amino acid
changes and fold improvement over Sr.KAH mutant #3 activity.

| Sr.KAH sequence | Fold | Percent |
|---|---|---|
| variations compared to | improvement over | improvement over |
| Sr.KAH mutant #3 | Sr.KAH mutant #3 | Sr.KAH mutant #3 |
| Sr.KAH mutant #3 | 1.00 | 0 |
| E492C | 1.17 | 17 |
| M427A | 1.18 | 18 |
| G306D | 1.19 | 19 |
| V333C | 1.19 | 19 |
| D191L | 1.21 | 21 |
| I40S | 1.21 | 21 |
| L445I | 1.21 | 21 |
| S114A | 1.23 | 23 |
| D191Y | 1.26 | 26 |
| D191F | 1.35 | 35 |
| L497R | 1.46 | 46 |
| G306L | 1.48 | 48 |
| N29G | 1.8 | 80 |
| T167G | 2.09 | 109 |
| T164S | 2.23 | 123 |
| Q415H | 2.6 | 160 |
| T89A | 2.71 | 171 |
| L13D | 3.11 | 211 |
| R258T | 3.65 | 265 |
| L13V | 4.28 | 328 |

Twenty mutations were identified that lead to improvements in activity, ranging from 17% to 4-fold over Sr.KAH mutant #3. The best mutant from this site directed saturation mutagenesis NNT library (L13V) provides 4.3-fold improvement of in vivo KAH activity comparing to Sr.KAH mutant #3 (which is already improved 5-fold over wild type Sr.KAH, Example 8). Interestingly, this conservative mutation (both leucine and valine have small hydrophobic side chains) is at the N-terminus of the protein where alterations via domain swap had significant effects on protein activity (Examples 9-12). Another beneficial mutation, N29G also belongs in the N-terminal domain of Sr.KAH. It is also interesting that the composition of mutations that improve the activity of wild type Sr.KAH (Example 7) is very different from those improving the activity of Sr.KAH mutant #3 (this Example). Mutant #3 differs by six amino acids from wild type Sr.KAH, with G306L being the only mutation beneficial for both enzyme variants: top hit for wild type and 9th best for mutant #3 of Sr.KAH.

All publications, patents and patent applications cited in this specification are herein incorporated by reference as if each individual publication or patent application were specifically and individually indicated to be incorporated by reference. Although the claims have been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

1.-12. (canceled)

13. A heterologous kaurenoic acid hydroxylase polypeptide with residue numbers according to SEQ ID NO:1, comprising at least one of the following mutations: Y62H, T164R, L76V, Q415L, A60Y, S182C, T167N, Y52H, R266D, T167H, K100L, G306C, Q120N, L232H, G351R, L232S, I166S, W447C, I443Y, I166N, N355Y, A442G, G306N, G447F, G306I, or G306V.

14.-29. (canceled)

30. A heterologous polynucleotide encoding the polypeptide of claim 13.

31. A yeast host cell comprising the heterologous polypeptide of claim 13.

32. The yeast host cell of claim 31 that is capable of producing steviol.

33. The yeast host cell of claim 32 that is capable of producing one or more steviol glycosides.

34. The yeast host cell of claim 31 that is capable of producing RebA, RebB, RebD, RebE, or RebM.

35. The yeast host cell of claim 31 that is capable of producing RebM.

36. A yeast host cell comprising the heterologous polynucleotide of claim 30.

37. The yeast host cell of claim 36, further comprising one or more heterologous nucleic acids encoding one or more enzymes of a pathway for making a steviol glycoside.

38. The yeast host cell of claim 36, further comprising a heterologous nucleic acid encoding a geranylgeranyl diphosphate synthase.

39. The yeast host cell of claim 36, further comprising a heterologous nucleic acid encoding a copalyl diphosphate synthase.

40. The yeast host cell of claim 36, further comprising a heterologous nucleic acid encoding an ent-kaurene synthase.

41. The yeast host cell of claim 36, further comprising a heterologous nucleic acid encoding a kaurene oxidase.

42. The yeast host cell of claim 36, further comprising a heterologous nucleic acid encoding a cytochrome P450 reductase.

43. The yeast host cell of claim 36, further comprising one or more heterologous nucleic acids encoding one or more uridine 5′-diphosphate-dependent glycosyltransferases.

44. The yeast host cell of claim 36, further comprising one or more heterologous nucleic acids encoding a geranylgeranyl diphosphate synthase, a copalyl diphosphate synthase, a ent-kaurene synthase, a kaurene oxidase, a cytochrome P450 reductase, a UGT_{AD}, UGT74G1, UGT76G1, UGT85C2, and UGT91D.

45. (canceled)

46. The yeast host cell of claim 37, wherein the one or more heterologous nucleic acids encoding one or more enzymes of the pathway are under control of a single transcriptional regulator.

47. The yeast host cell of claim 37, wherein the one or more heterologous nucleic acids encoding one or more enzymes of the pathway are under control of multiple transcriptional regulators.

48.-49. (canceled)

50. The host cell of claim 31, wherein the yeast is Saccharomyces cerevisiae.

51.-53. (canceled)

54. A fermentation composition comprising:
(a) a yeast host cell comprising:
i. a heterologous nucleic acid encoding a kaurenoic acid hydroxylase polypeptide of claim 13, capable of converting kaurenoic acid to steviol; and
(b) steviol glycosides produced from the host cell.


SEQUENCE LISTING (40 sequences)

SEQ ID NO: 1 - protein, 500 aa, Stevia rebaudiana
  REGION 1..500: note = wild type Stevia rebaudiana kaurenoic acid hydroxylase
MEASYLYISILLLLASYLFTTQLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRSSSSPVTLITVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSAGTMEWAMSLLVNHPHVLKKAQAEIDRVIGNNRLIDESDIGNIPYIGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 2 - protein, 550 aa, synthetic construct
  REGION 1..550: note = Synthetic: ATR2(1a:72a):Sr.KAH(23a:500a)
MSSSSSSSTSMIDLMAAIIKGEPVIVSDPANASAYESVAAELSSMLIENRQFAMIVTTSIAVLIGCIVMLVWLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRSSSSPVTLITVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSAGTMEWAMSLLVNHPHVLKKAQAEIDRVIGNNRLIDESDIGNIPYIGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 3 - protein, 546 aa, synthetic construct
  REGION 1..546: note = Synthetic: ATR2(1a:50a):Sr.KAH(5a:500a)
MSSSSSSSTSMIDLMAAIIKGEPVIVSDPANASAYESVAAELSSMLIENRYLYISILLLLASYLFTTQLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRSSSSPVTLITVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSAGTMEWAMSLLVNHPHVLKKAQAEIDRVIGNNRLIDESDIGNIPYIGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 4 - skipped

SEQ ID NO: 5 - protein, 519 aa, synthetic construct
  REGION 1..519: note = Synthetic: Sr.KO(1a:23a):Sr.KAH(5a:500a)
MDAVTGLLTVPATAITIGGTAVAYLYISILLLLASYLFTTQLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRSSSSPVTLITVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSAGTMEWAMSLLVNHPHVLKKAQAEIDRVIGNNRLIDESDIGNIPYIGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 6 - protein, 513 aa, synthetic construct
  REGION 1..513: note = Synthetic: Sr.KO
MDAVTGLLTVPATAITIGGTAVALAVALIFWYLKSYTSARRSQSNHLPRVPEVPGVPLLGNLLQLKEKKPYMTFTRWAATYGPIYSIKTGATSMVVVSSNEIAKEALVTRFQSISTRNLSKALKVLTADKTMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEIFQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRRHSPVPIIPLRHVHEDTVLGGYHVPAGTELAVNIYGCNMDKNVWENPEEWNPERFMKENETIDFQKTMAFGGGKRVCAGSLQALLTASIGIGRMVQEFEWKLKDMTQEEVNTIGLTTQMLRPLRAIIKPRI

SEQ ID NO: 7 - protein, 320 aa, synthetic construct
  REGION 1..320: note = Synthetic: Bt.GGPPS
MLTSSKSIESFPKNVQPYGKHYQNGLEPVGKSQEDILLEPFHYLCSNPGKDVRTKMIEAFNAWLKVPKDDLIVITRVIEMLHSASLLIDDVEDDSVLRRGVPAAHHIYGTPQTINCANYVYFLALKEIAKLNKPNMITIYTDELINLHRGQGMELFWRDTLTCPTEKEFLDMVNDKTGGLLRLAVKLMQEASQSGTDYTGLVSKIGIHFQVRDDYMNLQSKNYADNKGFCEDLTEGKFSFPIIHSIRSDPSNRQLLNILKQRSSSIELKQFALQLLENTNTFQYCRDFLRVLEKEAREEIKLLGGNIMLEKIMDVLSVNE

SEQ ID NO: 8 - protein, 736 aa, synthetic construct
  REGION 1..736: note = Synthetic: Ent-Os.CDPS
MEHARPPQGGDDDVAASTSELPYMIESIKSKLRAARNSLGETTVSAYDTAWIALVNRLDGGGERSPQFPEAIDWIARNQLPDGSWGDAGMFIVQDRLINTLGCVVALATWGVHEEQRARGLAYIQDNLWRLGEDDEEWMMVGFEITFPVLLEKAKNLGLDINYDDPALQDIYAKRQLKLAKIPREALHARPTTLLHSLEGMENLDWERLLQFKCPAGSLHSSPAASAYALSETGDKELLEYLETAINNFDGGAPCTYPVDNFDRLWSVDRLRRLGISRYFTSEIEEYLEYAYRHLSPDGMSYGGLCPVKDIDDTAMAFRLLRLHGYNVSSSVFNHFEKDGEYFCFAGQSSQSLTAMYNSYRASQIVFPGDDDGLEQLRAYCRAFLEERRATGNLRDKWVIANGLPSEVEYALDFPWKASLPRVETRVYLEQYGASEDAWIGKGLYRMTLVNNDLYLEAAKADFTNFQRLSRLEWLSLKRWYIRNNLQAHGVTEQSVLRAYFLAAANIFEPNRAAERLGWARTAILAEAIASHLRQYSANGAADGMTERLISGLASHDWDWRESNDSAARSLLYALDELIDLHAFGNASDSLREAWKQWLMSWTNESQGSTGGDTALLLVRTIEICSGRHGSAEQSLKNSEDYARLEQIASSMCSKLATKILAQNGGSMDNVEGIDQEVDVEMKELIQRVYGSSSNDVSSVTRQTFLDVVKSFCYVAHCSPETIDGHISKVLFEDVN

SEQ ID NO: 9 - protein, 757 aa, synthetic construct
  REGION 1..757: note = Synthetic: Ent-Pg.KS
MKREQYTILNEKESMAEELILRIKRMFSEIENTQTSASAYDTAWVAMVPSLDSSQQPQFPQCLSWIIDNQLLDGSWGIPYLIIKDRLCHTLACVIALRKWNAGNQNVETGLRFLRENIEGIVHEDEYTPIGFQIIFPAMLEEARGLGLELPYDLTPIKLMLTHREKIMKGKAIDHMHEYDSSLIYTVEGIHKIVDWNKVLKHQNKDGSLFNSPSATACALMHTRKSNCLEYLSSMLQKLGNGVPSVYPINLYARISMIDRLQRLGLARHFRNEIIHALDDIYRYWMQRETSREGKSLTPDIVSTSIAFMLLRLHGYDVPADVFCCYDLHSIEQSGEAVTAMLSLYRASQIMFPGETILEEIKTVSRKYLDKRKENGGIYDHNIVMKDLRGEVEYALSVPWYASLERIENRRYIDQYGVNDTWIAKTSYKIPCISNDLFLALAKQDYNICQAIQQKELRELERWFADNKFSHLNFARQKLIYCYFSAAATLFSPELSAARVVWAKNGVITTVVDDFFDVGGSSEEIHSFVEAVRVWDEAATDGLSENVQILFSALYNTVDEIVQQAFVFQGRDISIHLREIWYRLVNSMMTEAQWARTHCLPSMHEYMENAEPSIALEPIVLSSLYFVGPKLSEEIICHPEYYNLMHLLNICGRLLNDIQGCKREAHQGKLNSVTLYMEENSGTTMEDAIVYLRKTIDESRQLLLKEVLRPSIVPRECKQLHWNMMRILQLFYLKNDGFTSPTEMLGYVNAVIVDPIL

SEQ ID NO: 10 - protein, 499 aa, synthetic construct
  REGION 1..499: note = Synthetic: Ps.KO
MDTLTLSLGFLSLFLFLFLLKRSTHKHSKLSHVPVVPGLPVIGNLLQLKEKKPHKTFTKMAQKYGPIFSIKAGSSKIIVLNTAHLAKEAMVTRYSSISKRKLSTALTILTSDKCMVAMSDYNDFHKMVKKHILASVLGANAQKRLRFHREVMMENMSSKFNEHVKTLSDSAVDFRKIFVSELFGLALKQALGSDIESIYVEGLTATLSREDLYNTLVVDFMEGAIEVDWRDFFPYLKWIPNKSFEKKIRRVDRQRKIIMKALINEQKKRLTSGKELDCYYDYLVSEAKEVTEEQMIMLLWEPIIETSDTTLVTTEWAMYELAKDKNRQDRLYEELLNVCGHEKVTDEELSKLPYLGAVFHETLRKHSPVPIVPLRYVDEDTELGGYHIPAGSEIAINIYGCNMDSNLWENPDQWIPERFLDEKYAQADLYKTMAFGGGKRVCAGSLQAMLIACTAIGRLVQEFEWELGHGEEENVDTMGLTTHRLHPLQVKLKPRNRIY

SEQ ID NO: 11 - protein, 711 aa, synthetic construct
  REGION 1..711: note = Synthetic: At.CPR
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 12 - protein, 481 aa, synthetic construct
  REGION 1..481: note = Synthetic: UGT85C2
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 13 - protein, 460 aa, synthetic construct
  REGION 1..460: note = Synthetic: UGT74G1
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 14 - protein, 485 aa, synthetic construct
  REGION 1..485: note = Synthetic: UGT91D_like3
MYNVTYHQNSKAMATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 15 - protein, 458 aa, synthetic construct
  REGION 1..458: note = Synthetic: UGT76G1
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 16 - protein, 436 aa, synthetic construct
  REGION 1..436: note = Synthetic: UGT40087
MDASDSSPLHIVIFPWLAFGHMLASLELAERLAARGHRVSFVSTPRNISRLRPVPPALAPLIDFVALPLPRVDGLPDGAEATSDIPPGKTELHLKALDGLAAPFAAFLDAACADGSTNKVDWLFLDNFQYWAAAAAADHKIPCALNLTFAASTSAEYGVPRVEPPVDGSTASILQRFVLTLEKCQFVIQRACFELEPEPLPLLSDIFGKPVIPYGLVPPCPPAEGHKREHGNAALSWLDKQQPESVLFIALGSEPPVTVEQLHEIALGLELAGTTFLWALKKPNGLLLEADGDILPPGFEERTRDRGLVAMGWVPQPIILAHSSVGAFLTHGGWASTIEGVMSGHPMLFLTFLDEQRINAQLIERKKAGLRVPRREKDGSYDRQGIAGAIRAVMCEEESKSVFAANAKKMQEIVSDRNCQEKYIDELIQRLGSFEK

SEQ ID NO: 17-20 - skipped (4 entries)

SEQ ID NO: 21 - protein, 544 aa, synthetic construct
  REGION 1..544: note = Synthetic: Aa.CPR(1a:66a):Sr.KAH_mutant#10(23a:500a)
MQSTTSVKLSPFDLMTALLNGKVSFDTSNTSDTNIPLAVFMENRELLMILTTSVAVLIGCVVVLVWLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRDSSSPVTLRTVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSALTLEWAMSLLLNHPHVLKKAQAEIDRVIGNNRLIDESDIGNIPYLGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 22 - protein, 550 aa, synthetic construct
  REGION 1..550: note = Synthetic: ATR2(1a:72a):Sr.KAH_mutant#10(23a:500a)
MSSSSSSSTSMIDLMAAIIKGEPVIVSDPANASAYESVAAELSSMLIENRQFAMIVTTSIAVLIGCIVMLVWLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRDSSSPVTLRTVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSALTLEWAMSLLLNHPHVLKKAQAEIDRVIGNNRLIDESDIGNIPYLGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 23 - skipped

SEQ ID NO: 24 - protein, 544 aa, synthetic construct
  REGION 1..544: note = Synthetic: Aa.CPR(1a:66a):Sr.KAH_mutant#3(23a:500a)
MQSTTSVKLSPFDLMTALLNGKVSFDTSNTSDTNIPLAVFMENRELLMILTTSVAVLIGCVVVLVWLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLLRKLRDSSSPVTLRTVFYALTLNVIMRMISGKRYFDSGDRELEEEGKRFREILDETLLLAGASNVGDYLPILNWLGVKSDEKKLIALQKKRDDFFQGLIEQVRKSRGAKVGKGRKTMIELLLSLQESEPEYYTDAMIRSFVLGLLAAGSDTSAGTMEWAMSLLVNHPHVLKKAQAEIDRVVGNNRLIDESDIGNIPYLGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 25 - protein, 550 aa, synthetic construct
  REGION 1..550: note = Synthetic: ATR2(1a:72a):Sr.KAH_mutant#3(23a:500a)
MSSSSSSSTSMIDLMAAIIKGEPVIVSDPANASAYESVAAELSSMLIENRQFAMIVTTSIAVLIGCIVMLVWLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLLRKLRDSSSPVTLRTVFYALTLNVIMRMISGKRYFDSGDRELEEEGKRFREILDETLLLAGASNVGDYLPILNWLGVKSDEKKLIALQKKRDDFFQGLIEQVRKSRGAKVGKGRKTMIELLLSLQESEPEYYTDAMIRSFVLGLLAAGSDTSAGTMEWAMSLLVNHPHVLKKAQAEIDRVVGNNRLIDESDIGNIPYLGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 26 - protein, 523 aa, synthetic construct
  REGION 1..523: note = Synthetic: ALG11(1a:45a):Sr.KAH_mutant#10(23a:500a)
MGSAWTNYNFEEVKSHFGFKKYVVSSLVLVYGLIKVLTWIFRQWVLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRDSSSPVTLRTVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSALTLEWAMSLLLNHPHVLKKAQAEIDRVIGNNRLIDESDIGNIPYLGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 27 - protein, 529 aa, synthetic construct
  REGION 1..529: note = Synthetic: ERG11(1a:51a):Sr.KAH_mutant#10(23a:500a)
MSATKSIVGEALEYVNIGLSHFLALPLAQRISLIIIIPFIYNIVWQLLYSLLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRDSSSPVTLRTVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSALTLEWAMSLLLNHPHVLKKAQAEIDRVIGNNRLIDESDIGNIPYLGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 28 - protein, 534 aa, synthetic construct
  REGION 1..534: note = Synthetic: ERG11(1a:80a):Sr.KAH_mutant#10(47a:500a)
MSATKSIVGEALEYVNIGLSHFLALPLAQRISLIIIIPFIYNIVWQLLYSLRKDRPPLVFYWIPWVGSAVVYGMKPYEFFLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRDSSSPVTLRTVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSALTLEWAMSLLLNHPHVLKKAQAEIDRVIGNNRLIDESDIGNIPYLGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 29 - protein, 597 aa, synthetic construct
  REGION 1..597: note = Synthetic: NUS1(1a:119a):Sr.KAH_mutant#10(23a:500a)
MPTMIKKDDKAMEPPNEKPHRKIERDDVPESSNHIPPPESGVLKGGKVNSKTRALKAVTSIIADADENPQKKVNNETNGVQKQKTEDLSKRIGKFEYLFYKFLLVLLYICFGLFRYGQYLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRDSSSPVTLRTVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSALTLEWAMSLLLNHPHVLKKAQAEIDRVIGNNRLIDESDIGNIPYLGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 30 - protein, 540 aa, synthetic construct
  REGION 1..540: note = Synthetic: RCR1(1a:62a):Sr.KAH_mutant#10(23a:500a)
MGLISYENEAINEVKKADNHHVSKFVTSYYGPSSSSWQSGRWILFVLFVAAVILIILFTFVALRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRDSSSPVTLRTVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSALTLEWAMSLLLNHPHVLKKAQAEIDRVIGNNRLIDESDIGNIPYLGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 31 - protein, 528 aa, synthetic construct
  REGION 1..528: note = Synthetic: SEC66(1a:50a):Sr.KAH_mutant#10(23a:500a)
MSEFNETKFSNNGTFFETEEPIVETKSISVYTPLIYVFILVVSLVMFASSLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRDSSSPVTLRTVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSALTLEWAMSLLLNHPHVLKKAQAEIDRVIGNNRLIDESDIGNIPYLGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 32 - protein, 529 aa, synthetic construct
  REGION 1..529: note = Synthetic: UBP1(1a:52a):Sr.KAH_mutant#10(23a:500a)
MDLFIESKINSLLQFLFGSRQDFLRNFKTWSNNNNNLSIYLLIFGIVVFFYLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRDSSSPVTLRTVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSALTLEWAMSLLLNHPHVLKKAQAEIDRVIGNNRLIDESDIGNIPYLGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 33 - protein, 704 aa, Artemisia annua
  REGION 1..704: note = wild type Artemisia annua cytochrome P450 reductase Aa.CPR
MQSTTSVKLSPFDLMTALLNGKVSFDTSNTSDTNIPLAVFMENRELLMILTTSVAVLIGCVVVLVWRRSSSAAKKAAESPVIVVPKKVTEDEVDDGRKKVTVFFGTQTGTAEGFAKALVEEAKARYEKAVFKVIDLDDYAAEDDEYEEKLKKESLAFFFLATYGDGEPTDNAARFYKWFTEGEEKGEWLDKLQYAVFGLGNRQYEHFNKIAKVVDEKLVEQGAKRLVPVGMGDDDQCIEDDFTAWKELVWPELDQLLRDEDDTSVATPYTAAVAEYRVVFHDKPETYDQDQLTNGHAVHDAQHPCRSNVAVKKELHSPLSDRSCTHLEFDISNTGLSYETGDHVGVYVENLSEVVDEAEKLIGLPPHTYFSVHADNEDGTPLGGASLPPPFPPCTLRKALASYADVLSSPKKSALLALAAHATDSTEADRLKFLASPAGKDEYAQWIVASHRSLLEVMEAFPSAKPPLGVFFASVAPRLQPRYYSISSSPRFAPNRIHVTCALVYEQTPSGRVHKGVCSTWMKNAVPMTESQDCSWAPIYVRTSNFRLPSDPKVPVIMIGPGTGLAPFRGFLQERLAQKEAGTELGTAILFFGCRNRKVDFIYEDELNNFVETGALSELVTAFSREGATKEYVQHKMTQKASDIWNLLSEGAYLYVCGDAKGMAKDVHRTLHTIVQEQGSLDSSKAELYVKNLQMAGRYLRDVW

SEQ ID NO: 34 - protein, 712 aa, Arabidopsis thaliana
  REGION 1..712: note = wild type Arabidopsis thaliana cytochrome P450 reductase ATR2
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKVDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 35 - protein, 548 aa, Saccharomyces cerevisiae
  REGION 1..548: note = wild type Saccharomyces cerevisiae Alg11
  VARIANT 48: note = Xaa can be any naturally occurring amino acid
  VARIANT 75: note = Xaa can be any naturally occurring amino acid
MGSAWTNYNFEEVKSHFGFKKYVVSSLVLVYGLIKVLTWIFRQWVYSXLNPFSKKSSLLNRAVASCGEKNVKVFXFFHPYCNAGGGGEKVLWKAVDITLRKDAKNVIVIYSGDFVNGENITPENILNNVKAKFDYDLDSDRIFFISLKLRYLVDSSTWKHFTLIGQAIGSMILAFESIIQCPPDIWIDTMGYPFSYPIIARFLRRIPIVTYTHYPIMSKDMLNKLFKMPKKGIKGYGKILYWKVFMLIYQSIGSKIDIVITNSTWTNNHIKQIWQSNTCKIIYPPCSTEKLVDWKQKFGTAKGERLNQAIVLAQFRPEKRHKLIIESFATFLKNLPDSVSPIKLIMAGSTRSKQDENYVKSLQDWSENVLKIPKHLISFEKNLPFDKIEILLNKSTFGVNAMWNEHFGIAVVEYMASGLIPIVHASAGPLLDIVTPWDANGNIGKAPPQWELQKKYFAKLEDDGETTGFFFKEPSDPDYNTTKDPLRYPNLSDLFLQITKLDYDCLRVMGARNQQYSLYKFSDLKFDKDWENFVLNPICKLLEEEERG

SEQ ID NO: 36 - protein, 530 aa, Saccharomyces cerevisiae
  REGION 1..530: note = wild type Saccharomyces cerevisiae Erg11
MSATKSIVGEALEYVNIGLSHFLALPLAQRISLIIIIPFIYNIVWQLLYSLRKDRPPLVFYWIPWVGSAVVYGMKPYEFFEECQKKYGDIFSFVLLGRVMTVYLGPKGHEFVFNAKLADVSAEAAYAHLTTPVFGKGVIYDCPNSRLMEQKKFVKGALTKEAFKSYVPLIAEEVYKYFRDSKNFRLNERTTGTIDVMVTQPEMTIFTASRSLLGKEMRAKLDTDFAYLYSDLDKGFTPINFVFPNLPLEHYRKRDHAQKAISGTYMSLIKERRKNNDIQDRDLIDSLMKNSTYKDGVKMTDQEIANLLIGVLMGGQHTSAATSAWILLHLAERPDVQQELYEEQMRVLDGGKKELTYDLLQEMPLLNQTIKETLRMHHPLHSLFRKVMKDMHVPNTSYVIPAGYHVLVSPGYTHLRDEYFPNAHQFNIHRWNNDSASSYSVDEEVDYGFGAISKGVSSPYLPFGGGRHRCIGEHFAYCQLGVLMSIFIRTLKWHYPEGKTVPPPDFTSMVTLPTGPAKIIWEKRNPEQKI

SEQ ID NO: 37 - protein, 375 aa, Saccharomyces cerevisiae
  REGION 1..375: note = wild type Saccharomyces cerevisiae Nus1p
MPTMIKKDDKAMEPPNEKPHRKIERDDVPESSNHIPPPESGVLKGGKVNSKTRALKAVTSIIADADENPQKKVNNETNGVQKQKTEDLSKRIGKFEYLFYKFLLVLLYICFGLFRYGQYQYHKMKLRIFSIIYNHAYTPQLIRQDVIPLKKIPKRLAAILEVKPVGDVGGGVTGLLNDASEIVCWTVSAGIKHLMLYDYDGILQRNVPELRMEIHSNLAKYFGPAHVPNYAVKIPHSNKIFYNLDGIETETDVGNEIEANQEKDKIAIEISLLSNRDGRETIVDLTKTMAELCAVNELSVSDITMDLVDSELKQLVGPEPDLLLYFGPSLDLQGFPPWHIRLTEFYWEKDNNEVIYSVFIRGLRQYAGCKVNVGK

SEQ ID NO: 38 - protein, 213 aa, Saccharomyces cerevisiae
  REGION 1..213: note = wild type Saccharomyces cerevisiae Rcr1
MGLISYENEAINEVKKADNHHVSKFVTSYYGPSSSSWQSGRWILFVLFVAAVILIILFTFVANRRRRRMGRAPIRGTAWLTPPSYRQSQQQYTGTVQQRTDDYVPEYTETANEHDLGYYDQRGEFHPNDKAAYVAPPPLVQECSSESVNSLERPPAAVIHQANSLDTDYGLTRPSNGRVPAVSDTVEQLERLPGGTTTQEINPPERAKVNARS

SEQ ID NO: 39 - protein, 206 aa, Saccharomyces cerevisiae
  REGION 1..206: note = wild type Saccharomyces cerevisiae Sec66p
MSEFNETKFSNNGTFFETEEPIVETKSISVYTPLIYVFILVVSLVMFASSYRKKQAKKISEQPSIFDENDAHDLYFQIKEMSENEKIHEKVLKAALLNRGAESVRRSLKLKELAPQINLLYKNGSIGEDYWKRFETEVKLIELEFKDTLQEAERLQPGWVQLFVMVCKEICFNQALSRRYQSTLKRKEVCIKEWELKINNDGRLVN

SEQ ID NO: 40 - protein, 811 aa, Saccharomyces cerevisiae
  REGION 1..811: note = wild type Saccharomyces cerevisiae Ubp1p
MDLFIESKINSLLQFLFGSRQDFLRNFKTWSNNNNNLSIYLLIFGIVVFFYKKPDHLNYIVESVSEMTTNFRNSNSLSRWLPRSKFTHLDEEILKRGGFIAGLVNDGNTCFMNSVLQSLASSRELMEFLDNNVIRTYEEIEQNEHNEEENGQESAQDEATHKKNTRKGGKVYGKHKKKLNRKSSSKEDEEKSQEPDITFSVALRDLLSALNAKYYRDKPYFKTNSLLRAMSKSPRKNILLGYDQEDAQEFFQNILAELESNVKSLNTEKLDTTPVAKSELPDDALVGQLNLGEVGTVYIPTEQIDPNSILHDKSIQNFTPFKLMTPLDGITAERIGCLQCGENGGIRYSVFSGLSLNLPNENIGSTLKLSQLLSDWSKPEIIEGVECNRCALTAAHSHLFGQLKEFEKKPEGSIPEKLINAVKDRVHQIEEVLAKPVIDDEDYKKLHTANMVRKCSKSKQILISRPPPLLSIHINRSVFDPRTYMIRKNNSKVLFKSRLNLAPWCCDINEINLDARLPMSKKEKAAQQESSEDENIGGEYYTKLHERFEQEFEDSEEEKEYDDAEGNYASHYNHTKDISNYDPLNGEVDGVTSDDEDEYIEETDALGNTIKKRIIEHSDVENENVKDNEELQEIDNVSLDEPKINVEDQLETSSDEEDVIPAPPINYARSFSTVPATPLTYSLRSVIVHYGTHNYGHYIAFRKYRGCWWRISDETVYVVDEAEVLSTPGVFMLFYEYDFDEETGKMKDDLEAIQSNNEEDDEKEQEQEQKGVQEPKESQEQGEGEEQEEGQEQMKFERTEDHRDISGKDVN